SEQUENCE LISTING (74 sequences)

SEQ ID NO: 1 - protein, 10 aa, Homo sapiens
GGTLSNYVIT

SEQ ID NO: 2 - protein, 8 aa, Homo sapiens
FIPTFRTA

SEQ ID NO: 3 - protein, 11 aa, Homo sapiens
ARGPLSRGYYD

SEQ ID NO: 4 - protein, 10 aa, Homo sapiens
GGTFNIYTVN

SEQ ID NO: 5 - protein, 8 aa, Homo sapiens
IIPMLGSA

SEQ ID NO: 6 - protein, 17 aa, Homo sapiens
ARSLPREMFFRDDAFDI

SEQ ID NO: 7 - protein, 10 aa, Homo sapiens
GTSFNRYTFA

SEQ ID NO: 8 - protein, 8 aa, Homo sapiens
IIPIVGLT

SEQ ID NO: 9 - protein, 22 aa, Homo sapiens
ARDKVEFWLGEIAPNKFDVFDL

SEQ ID NO: 10 - protein, 10 aa, Homo sapiens
GDTFSSYSIH

SEQ ID NO: 11 - protein, 8 aa, Homo sapiens
IIPIVGLV

SEQ ID NO: 12 - skipped

SEQ ID NO: 13 - protein, 10 aa, Homo sapiens
GDTFSSYSIH

SEQ ID NO: 14 - protein, 8 aa, Homo sapiens
IIPIVGLV

SEQ ID NO: 15 - protein, 23 aa, Homo sapiens
ARASLGCPNGVCHGPRAADYFDY

SEQ ID NO: 16 - protein, 9 aa, Homo sapiens
GTFSLNSLS

SEQ ID NO: 17 - protein, 8 aa, Homo sapiens
IIPIVGIA

SEQ ID NO: 18 - protein, 18 aa, Homo sapiens
ARELYSGTGFFRLEALDY

SEQ ID NO: 19 - protein, 10 aa, Homo sapiens
GGTLSNYVIT

SEQ ID NO: 20 - protein, 8 aa, Homo sapiens
FIPTFRTA

SEQ ID NO: 21 - protein, 11 aa, Homo sapiens
ARGPLSRGYYD

SEQ ID NO: 22 - protein, 10 aa, Homo sapiens
GGTLSNYVIT

SEQ ID NO: 23 - protein, 8 aa, Homo sapiens
FIPTFRTA

SEQ ID NO: 24 - protein, 11 aa, Homo sapiens
ARGPLSRGYYD

SEQ ID NO: 25 - protein, 10 aa, Homo sapiens
GGTLSNYVIT

SEQ ID NO: 26 - protein, 8 aa, Homo sapiens
FIPTFRTA

SEQ ID NO: 27 - protein, 11 aa, Homo sapiens
ARGPLSRGYYD

SEQ ID NO: 28 - protein, 9 aa, Homo sapiens
NNIGSKSVH

SEQ ID NO: 29 - protein, 6 aa, Homo sapiens
YDDSDR

SEQ ID NO: 30 - protein, 12 aa, Homo sapiens
QVWDSSSVVFGG

SEQ ID NO: 31 - protein, 6 aa, Homo sapiens
QSVSSN

SEQ ID NO: 32 - protein, 3 aa, Homo sapiens
GAS

SEQ ID NO: 33 - protein, 8 aa, Homo sapiens
QQYGSSPV

SEQ ID NO: 34 - protein, 12 aa, Homo sapiens
QSIFFSSNSKDY

SEQ ID NO: 35 - protein, 3 aa, Homo sapiens
WAS

SEQ ID NO: 36 - protein, 9 aa, Homo sapiens
QQYFDTPQI

SEQ ID NO: 37 - protein, 6 aa, Homo sapiens
QSISSW

SEQ ID NO: 38 - protein, 3 aa, Homo sapiens
DAS

SEQ ID NO: 39 - protein, 8 aa, Homo sapiens
QQYDNLPR

SEQ ID NO: 40 - protein, 7 aa, Homo sapiens
QRVPSSS

SEQ ID NO: 41 - protein, 3 aa, Homo sapiens
GAS

SEQ ID NO: 42 - protein, 8 aa, Homo sapiens
QQYGSSLI

SEQ ID NO: 43 - protein, 6 aa, Homo sapiens
QSVSSY

SEQ ID NO: 44 - protein, 3 aa, Homo sapiens
DAS

SEQ ID NO: 45 - protein, 7 aa, Homo sapiens
QQYGSPQ

SEQ ID NO: 46 - protein, 9 aa, Homo sapiens
NNIGSKNVH

SEQ ID NO: 47 - protein, 6 aa, Homo sapiens
YRDSNR

SEQ ID NO: 48 - protein, 12 aa, Homo sapiens
QVWDSSTAVFGG

SEQ ID NO: 49 - protein, 9 aa, Homo sapiens
NNIGSKSVH

SEQ ID NO: 50 - protein, 6 aa, Homo sapiens
YDDSDR

SEQ ID NO: 51 - protein, 12 aa, Homo sapiens
QVWDSSSVVFGG

SEQ ID NO: 52 - protein, 9 aa, Homo sapiens
DKLGDKYAS

SEQ ID NO: 53 - protein, 6 aa, Homo sapiens
YQDNKR

SEQ ID NO: 54 - protein, 12 aa, Homo sapiens
QAWDSNTGVFGT

SEQ ID NO: 55 - protein, 123 aa, Homo sapiens
QVQLVQSGAEVKKPGSSVKVSCEASGGTLSNYVITWVRQAPGQGLEWMGGFIPTFRTAMYAQGFQGRVTITADESTSIAYMELTNLRSEDTAVYYCARGPLSRGYYDYWGQGTLVTVSSGSTG

SEQ ID NO: 56 - protein, 127 aa, Homo sapiens
QVQLVQSGSEVKKPGSSVKVSCKASGGTFNIYTVNWVRQAPGQGLEWMGRIIPMLGSADYAQRFQGRVTISADKSTSTAYMELRNLKSEDTAVYYCARSLPREMFFRDDAFDIWGQGTMVTVSSGST

SEQ ID NO: 57 - protein, 159 aa, Homo sapiens
LPREMFFRDDAFDIWGQGTMVTVSSGSTQVQLVQSGAEVKKPGSSVKVSCKASGTSFNRYTFAWVRQAPGQGLEWVGTIIPIVGLTNYAEKFQGRVSITADKSTSTTYMELSSLRSEDTAVYFCARKVEFWLGEIAPNKFDVFDLWGQGTMVTVSSGST

SEQ ID NO: 58 - protein, 132 aa, Homo sapiens
EVQLVQSGAEVKKPGSSVKVSCKASGDTFSSYSIHWVRQAPGQGLEWMGRIIPIVGLVDYAQMFQGRVTISADRSTNTVYMDLSNLRSEDTAIYYCARSLGCPNGVCHGPRAADYFDYWGQGTLVTVSSGST

SEQ ID NO: 59 - protein, 132 aa, Homo sapiens
QVQLVESGAEVKKPGSSVKVSCKASGDTFSSYSIHWVRQAPGQGLEWMGRIIPIVGLVDYAQMFQGRVTISADRSTNTVYMDLSNLRSEDTAYYCARASLGCPNGVCHGPRAADYFDYWGQGTLVTVSSGST

SEQ ID NO: 60 - protein, 127 aa, Homo sapiens
QVQLVQSGAEVKKPGSSVKVSCKASGGTFSLNSLSWVRQAPGQGLEWMGTIIPIVGIANYAQKFQGRVTITADKSTGTAYMDLSSLSDDTAVYFCARELYSGTGFFRLEALDYWGQGTLVTVSSGST

SEQ ID NO: 61 - protein, 119 aa, Homo sapiens
QVQLQQSGAEVKKPGSSVKVSCEASGGTLSNYVITWVRQAPGQGLEWMGGFIPTFRTAMYAQGFQGRVTITADESTSIAYMELTNLRSEDTAVYYCARGPLSRGYYDYWGQGTLVTVSS

SEQ ID NO: 62 - protein, 119 aa, Homo sapiens
QVQLVQSGAEVKKPGSSVKVSCEASGGTLSNYVITWVRQAPGQGLEWMGGFIPTFRTAMYAQGFQGRVTITADESTSIAYMELTNLRSEDTAVYYCARGPLSRGYYDYWGQGTLVTVSS

SEQ ID NO: 63 - protein, 119 aa, Homo sapiens
QVTLKESGAEVKKPGSSVKVSCEASGGTLSNYVITWVRQAPGQGLEWMGGFIPTFRTAMYAQGFQGRVTITADESTSIAYMELTNLRSEDTAVYYCARGPLSRGYYDYWGQGTLVTVSS

SEQ ID NO: 64 - protein, 124 aa, Homo sapiens
  misc_feature (101)..(101): Xaa can be any naturally occurring amino acid
SSYVLTQPPSVSVAPGKTARITCGGNNIGSKSVHWYQQKPGQAPVLVVYDDSDRPSGIPERFSGSNSGNTATLTISRVEAGDEADYYCQVWDSSSVVFGGXTKLTVLAAAGGPFESKPIPNPLL

SEQ ID NO: 65 - protein, 124 aa, Homo sapiens
ETTLTQSPGTLSLSPGERATLSCRASQSVSSNLAWYQQKPGQAPRLLIYGASTRATGIPDRFSGSGSGTDFTLTISRLEPDDFVVYYCQQYGSSPVTFGQGTRLEIKRAAAGGPFEGKPIPNPL

SEQ ID NO: 66 - protein, 122 aa, Homo sapiens
EIVLTQSPDSLAVSLGERATIHCKSSQSIFFSSNSKDYLAWYQQKPGQPPKLLIYWASTRDSGVPDRFSGSGSGTDFTLTITGLQADDAAVYYCQQFDTPQITFGGGTKLEIKRAAAGGPFE

SEQ ID NO: 67 - protein, 124 aa, Homo sapiens
  misc_feature (117)..(117): Xaa can be any naturally occurring amino acid
EMTLTQSPSSLSASVGDRVTITCRASQSISSWLAWYQQKPGKAPKLLIYDASNLETGVPSRFSGSGSGTDFTFTISSLQPEDIATYYCQQYDNLPRTFGQGTKVDIKRAAAGGPFEXKPIPNPL

SEQ ID NO: 68 - protein, 125 aa, Homo sapiens
  misc_feature (117)..(117): Xaa can be any naturally occurring amino acid
  misc_feature (119)..(119): Xaa can be any naturally occurring amino acid
EIVLTQSPGTVSLFPGDRVTLSCRASQRVPSSSLAWYQQKPGQAPRLLLYGASSRATGIPDRFSGRGSGTDFALSISRLEPEDFAVYYCQQYGSSLITFGQGTRLEIKRAAAGGPFXGXPIPNPL

SEQ ID NO: 69 - protein, 122 aa, Homo sapiens
DIQMTQSPATLSLSPGERATLSCRASQSVSSYLAWYQQKPGQAPRLLIYDASNRATGIPARFIAGGSGTDFTLTISSLEPEDFAVYYCQQYGSPQTFGQGTKLEIKRAAAGGPFEGKPIPNP

SEQ ID NO: 70 - protein, 106 aa, Homo sapiens
SYELTQPLSVSVALGQTARITCGGNNIGSKNVHWYQQKPGQAPVLVIYRDSNRPSGIPERFSGSNSGNTATLTISRAQAGDEADYYCQVWDSSTAVFGGGTMLTVL

SEQ ID NO: 71 - protein, 106 aa, Homo sapiens
SYVLTQPPSVSVAPGKTARITCGGNNIGSKSVHWYQQKPGQAPVLVVYDDSDRPSGIPERFSGSNSGNTATLTISRVEAGDEADYYCQVWDSSSVVFGGGTKLTVL

SEQ ID NO: 72 - protein, 106 aa, Homo sapiens
SYVLTQPPSVSVSPGQTARITCSGDKLGDKYASWYQQKPGQSPVLVIYQDNKRPSEIPARFSGSNSGNTATLTISGAQAMDEADYYCQAWDSNTGVFGTGTKLTVL

SEQ ID NO: 73 - protein, 116 aa, Homo sapiens
QVQLVQSGAEVKKPGSSVKVSCKASGGTFSYSITWVRQAPGQGLEWMGIIPIVGTAYAQFQGRVTITADKSTSTAYMELSNLRSEDTAVYYCARGPSDYFDYWGQGTLVTVSSGST

SEQ ID NO: 74 - protein, 108 aa, Homo sapiens
ELTQSPSVSVSPGERATITCRASQVSLAWYQQKPGQAPRLLIYASRSGIPDRFSGSGSGTDFTLTISRLEAEDAYYCQQYDSSVTFGGTKLEIKRAAAGGPFEPIPNP

(19) United States
(12) Patent Application Publication
(10) Pub. No.: US 2013/0084301 A1
(43) Pub. Date: Apr. 4, 2013

(54) Cluster of Neutralizing Antibodies to Hepatitis C Virus

(71) Applicants: Steven Foung, Stanford, CA (US); Zhen-Yong Keck, Redwood City, CA (US)

(72) Inventors: Steven Foung, Stanford, CA (US); Zhen-Yong Keck, Redwood City, CA (US)

(21) Appl. No.: 13/599,796

(22) Filed: Aug. 30, 2012

Related U.S. Application Data
(60) Provisional application No. 61/529,147, filed on Aug. 30, 2011.

(51) Int. Cl. C07K 16/10; A61K 39/29; A61K 39/42; A61K 45/06; G01N 33/569; G01N 33/68

(52) U.S. Cl. CPC C07K 16/10; G01N 33/56983; G01N 33/6854; A61K 39/42; A61K 45/06; A61K 39/29
USPC 424/161.1; 530/389.4; 530/388.3; 530/388.15; 536/23.53; 435/339; 435/5; 435/7.92; 506/9; 424/228.1

(57) ABSTRACT

Compositions and methods are provided relating to human anti-HCV E2 monoclonal antibodies. The antibodies of the invention bind to a conserved region of HCV E2 protein, and neutralize HCV influenza virus across multiple HCV genotypes. Embodiments of the invention include isolated antibodies and derivatives and fragments thereof, pharmaceutical formulations comprising one or more of the human anti-HCV monoclonal antibodies; and cell lines that produce these monoclonal antibodies.

### CROSS REFERENCE TO RELATED APPLICATIONS

Pursuant to 35 U.S.C. §119 (e), this application claims priority to the filing date of the U.S. Provisional Patent Application Ser. No. 61/529,147 filed Aug. 30, 2011; the disclosure of which is herein incorporated by reference.

### GOVERNMENT RIGHTS

This invention was made with Government support under contract A1081903 awarded by the National Institutes of Health. The Government has certain rights in this invention.

### BACKGROUND

End-stage liver disease caused by hepatitis C virus (HCV) is a leading indication of liver transplantation. However, reinfection with HCV occurs universally, and allograft failure due to reinfection is the most common cause of re-transplantation and death among HCV-infected liver transplant recipients. Over one third of recipients develops cirrhosis and needs re-transplantation by the fifth postoperative year. Combined treatment with (pegylated) interferon and ribavirin has poor tolerability and efficacy in liver transplant recipients, with only approximately 30% sustained virological clearance occurring in treated patients.

Recent advances in in vitro and in vivo HCV infection systems and increased understanding of HCV virology have led to the development of many HCV-specific small molecules with antiviral activity. Completion of Phase III studies with several protease inhibitors were recently presented with promising results. However, the potential for escape HCV mutants from these recently FDA approved protease inhibitors, the proviral effects of post-liver transplant immunosuppression on HCV biology, the diminished tolerability of interferon and ribavirin in the post-transplant setting, and the potential for interactions of new antivirals with immunosuppressive agents are likely to limit the utility of new antiviral therapies in liver transplant recipients for at least the medium term.

A model for HCV is available in liver transplantation for hepatitis B (HBV). While nucleotide and nucleoside analogs are well tolerated and effective for suppression of HBV, hepatitis B immunoglobulin (HBIg) is required and is a standard of care for prevention of post-liver transplant HBV infection. HBIg has moved HBV infected patients from the ranks of the not transplantable to ideal candidates for liver transplantation. Thus, an effective hepatitis C immunoglobulin is a possible cornerstone of preventing post-liver transplant HCV infection, even if more efficacious and well tolerated oral anti-HCV therapies are developed.

HCV can be classified into seven genetically distinct genotypes and further subdivided into a large number of subtypes, of which the seven major genotypes differ by approximately 30%, and the subtypes differ by 20%-25%, at the nucleotide level. A significant challenge for immunotherapeutic development is the identification of protective epitopes conserved in the majority of viral genotypes and subtypes. This problem is compounded by the fact that the envelope E1E2 glycoproteins, the natural targets for the neutralizing response, are two of the most variable proteins. The error-prone nature of the RNA-dependent RNA polymerase, together with the high HCV replicative rate in vivo, results in the production of viral quasispecies. Selected antibodies ideally should be broadly reactive to different HCV genotypes, each inhibiting at different steps of virus entry, and be synergistic in their ability to control virus infection.

Cross-competition analyses with human monoclonal antibodies (HMAbs) to conformational epitopes on HCV E2 delineate at least three immunogenic clusters of overlapping epitopes with distinct properties. Non-neutralizing HMAbs fall into one cluster, designated as domain A, while neutralizing HMAbs segregated into two clusters, designated as domains B and C. Antibodies within domains B and C mediate neutralization by inhibiting E2 binding to the required co-receptor CD81. Domain B antibodies mediate varying degrees of neutralization against HCV pseudotype particles (HCVpp) containing E1E2 glycoproteins of HCV genotypes 1 to 6, with some neutralizing all genotypes. Alanine scanning mutagenesis revealed that two conserved E2 residues (G530 and D535) are required for binding of all domain B HMAbs, while G523 or W529 are required for some but not all of these antibodies. G523, W529, G530 and D535 are also contact residues for HCV attachment to CD81. These findings, thus, suggest that domain B HMAbs exert potent and potentially broad neutralizing effects on HCV by competing with CD81 for binding to conserved residues on E2 that are important for viral entry. Consistent with this, broadly neutralizing HMAbs isolated from combinatorial libraries derived from B cells from individuals with chronic hepatitis C also recognize conformational epitopes containing these contact residues.

Treatment of HCV and the development of neutralizing antibodies that broadly react with HCV subtypes is of interest in the field. The present invention addresses this issue.

### SUMMARY OF THE INVENTION

Compositions and methods are provided relating to human anti-HCV E2 monoclonal antibodies. The antibodies of the invention bind to a conformational epitope in a conserved and essential region of HCV E2 protein, and neutralize HCV influenza virus across multiple HCV genotypes. Embodiments of the invention include isolated antibodies and derivatives and fragments thereof, pharmaceutical formulations comprising one or more of the human anti-HCV monoclonal antibodies; and cell lines that produce these monoclonal antibodies. Also provided are CDR amino acid sequences that confer the binding specificity of these monoclonal antibodies. These sequences and the cognate epitopes to which the monoclonal antibodies of the invention bind can be used to identify other antibodies that specifically bind and neutralize HCV; and immunotherapeutic methods for prevention of disease associated with HCV virus, including without limitation the neutralization of virus in association with liver transplantation. Therapies of interest include combination therapies with anti-HCV therapeutics such as monoclonal antibodies that specifically bind a different epitope than the antibodies of the invention, small molecule antivirals, interferon, and the like.

Antigenic compositions are provided, which comprise all or a portion of an HCV E2 protein in which specific highly immunodominant residues are masked, so as to generate an immune response to residues that are less immunodominant, but which are essential for virus function and therefore are less likely to be altered in virus escape mutation and selection. Such polypeptides are typically at least about 50 amino acids of contiguous E2 sequence, at least about 100 amino acids, at least about 200 amino acids, up to substantially all of the E2 protein. These antigens find use in screening assays, generation of monoclonal antibodies, and in vaccines. Residues of interest for masking include Y632 and D535, which can be masked by substituting the native amino acid with alanine, serine, etc.

The cluster of neutralizing human monoclonal antibodies disclosed herein bind to overlapping epitopes that are highly conserved among different HCV variants. These antibodies are designated herein HC-84 antibodies. Epitope mapping has demonstrated that contact residues between HCV E2 protein and HC-84 antibodies include the region of E2 amino acids 420-446 and 613-616. Antibodies of interest include antibodies that bind to an epitope which includes one or more of HCV E2 AA420-446 or HCV E2 AA613-616, and which epitope does not include either or both of HCV E2 AA530 or HCV E2 AA535.

Antibodies of interest include the provided HC-84.1; HC-84.20; HC-84.21; HC-84.22; HC-23; HC-84.24; HC-84.25; HC-84.26 and HC-84.27. An advantage of the monoclonal antibodies of the invention derives from the fact that they are encoded by a human polynucleotide sequence. Thus, in vivo use of the monoclonal antibodies of the invention for immunotherapy greatly reduces the problems of significant host immune response to the passively administered antibodies.

The human anti-HCV antibody may have a heavy chain variable region comprising the amino acid sequence of CDR1 and/or CDR2 and/or CDR3 of the provided human monoclonal human antibodies as provided herein; and/or a light chain variable region comprising the amino acid sequence of CDR1 and/or CDR2 and/or CDR3 of the provided human monoclonal human antibodies as provided herein. In other embodiments, the antibody comprises an amino acid sequence variant of one or more of the CDRs of the provided human antibodies, which variant comprises one or more amino acid insertion(s) within or adjacent to a CDR residue and/or deletion(s) within or adjacent to a CDR residue and/or substitution(s) of CDR residue(s) (with substitution(s) being the preferred type of amino acid alteration for generating such variants). Such variants will normally having a binding affinity for HCV E2 of at least about 10⁸, and will bind to the same epitope as an antibody having the amino acid sequence of at least one of HC-84.1; HC-84.20; HC-84.21; HC-84.22; HC-23; HC-84.24; HC-84.25; HC-84.26 and HC-84.27.

In some embodiments of the invention, antibodies compete for binding with HC-84.1; HC-84.20; HC-84.21; HC-84.22; HC-23; HC-84.24; HC-84.25; HC-84.26 and HC-84.27, e.g. compete for binding to HCV virus or polypeptides derived therefrom. Some antibodies are readily defined using the methods described herein.

Various forms of the antibodies are contemplated herein. For example, the anti-HCV antibody may be a full length antibody, e.g. having a human immunoglobulin constant region of any isotope, e.g. IgG1, IgG2a, IgG2b, IgG3, IgG4, IgA, etc. or an antibody fragment, e.g. a F(ab′)₂ fragment, and F(ab) fragment, etc. Furthermore, the antibody may be labeled with a detectable label, immobilized on a solid phase and/or conjugated with a heterologous compound.

Diagnostic and therapeutic uses for the antibody are contemplated. In one diagnostic application, the invention provides a method for determining the presence of HCV virus comprising exposing a sample suspected of containing the HCV virus to the anti-HCV antibody and determining binding of the antibody to the sample.

The invention further provides: isolated nucleic acid encoding the antibodies and variants; a vector comprising that nucleic acid, optionally operably linked to control sequences recognized by a host cell transformed with the vector; a host cell comprising that vector; a process for producing the antibody comprising culturing the host cell so that the nucleic acid is expressed and, optionally, recovering the antibody from the host cell culture (e.g. from the host cell culture medium). The invention also provides a composition comprising one or more of the human anti-HCV antibodies and a pharmaceutically acceptable carrier or diluent. This composition for therapeutic use is sterile and may be lyophilized, e.g. being provided as a pre-pack in a unit dose with diluent and delivery device, e.g. inhaler, syringe, etc.

Other aspects and features will be readily apparent to the ordinarily skilled artisan upon reading the present disclosure.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The invention is best understood from the following detailed description of exemplary embodiments when read in conjunction with the accompanying drawings. It is emphasized that, according to common practice, the various features of the drawings are not necessarily to-scale. On the contrary, the dimensions of the various features are arbitrarily expanded or reduced for clarity. Included in the drawings are the following figures:

FIG. 1A-1C. FIG. 1A is a graph depicting the neutralization of HCVcc1a by selected antibodies of the invention. FIG. 2B is a graph depicting the neutralization of HCVcc2a with selected antibodies of the invention. FIG. 1C provides the IC₅₀ for antibodies of the invention against HCV genotypes.

FIGS. 2A-2D. FIG. 2A is a table depicting binding of selected antibodies of the invention to different HCV genotypes. FIG. 2B is a graph depicting the binding of selected antibodies of the invention to H77c E1E2 protein. FIG. 2C is a graph depicting SPR kinetic binding; FIG. 2D is a table of affinity contacts of selected antibodies of the invention.

FIG. 3. FIG. 3A shows competitive binding of selected antibodies of the invention to domain A, B and C antibodies. FIG. 3B is a graph depicting inhibition of binding to CD81. FIG. 3C is a schematic showing relationship of antibodies to different HCV domains.

FIG. 4 is an overview of binding to alanine scanned mutations in regions 1, 2 and 3 of HCV E2 protein.

FIG. 5A-5B. (A) a summary of contact residues for selected antibodies of the invention. (B) a graph of binding HC84.21 to alanine substituted proteins at residues 441, 442 and 443.

FIG. 6 is a Summary of Escape Mutant Generation Status for HC84 related antibodies.

FIG. 7A provides the amino acid sequence of heavy chain CDR regions in selected antibodies of the invention. The amino acid sequence of heavy chain CDR1, 2 and 3, respectively of HC84.1 are set forth in SEQ ID NO:1, SEQ ID NO:2 and SEQ ID NO:3; similarly the CDR regions of HC84.20 are set forth as SEQ ID NO:4, SEQ ID NO:5 and SEQ ID NO:6; of HC-84.21 are set forth as SEQ ID NO:7, SEQ ID NO:8 and SEQ ID NO:9; HC-84.22 are set forth as SEQ ID NO:10, SEQ ID NO:11 and SEQ ID NO:12; HC-23 are set forth as SEQ ID NO:13, SEQ ID NO:14 and SEQ ID NO:15; HC-84.24 are set forth as SEQ ID NO:16, SEQ ID NO:17 and SEQ ID NO:18; HC-84.25 are set forth as SEQ ID NO:19, SEQ ID NO:20 and SEQ ID NO:21; HC-84.26 are set forth as SEQ ID NO:22, SEQ ID NO:23 and SEQ ID NO:24 and HC-84.27 are set forth as SEQ ID NO:25, SEQ ID NO:26 and SEQ ID NO:27.

FIG. 7B provides the amino acid sequence of light chain CDR regions in selected antibodies of the invention. The amino acid sequence of light chain CDR1, 2 and 3, respectively of HC84.1 are set forth in SEQ ID NO:28, SEQ ID NO:29 and SEQ ID NO:30; similarly the CDR regions of HC84.20 are set forth as SEQ ID NO:31, SEQ ID NO:32 and SEQ ID NO:33; of HC-84.21 are set forth as SEQ ID NO:34, SEQ ID NO:35 and SEQ ID NO:36; HC-84.22 are set forth as SEQ ID NO:37, SEQ ID NO:38 and SEQ ID NO:39; HC-23 are set forth as SEQ ID NO:40, SEQ ID NO:41 and SEQ ID NO:42; HC-84.24 are set forth as SEQ ID NO:43, SEQ ID NO:44 and SEQ ID NO:45; HC-84.25 are set forth as SEQ ID NO:46, SEQ ID NO:47 and SEQ ID NO:48; HC-84.26 are set forth as SEQ ID NO:49, SEQ ID NO:50 and SEQ ID NO:51 and HC-84.27 are set forth as SEQ ID NO:52, SEQ ID NO:53 and SEQ ID NO:54.

FIG. 8 provides an alignment of the sequences of the variable regions in selected antibodies of the invention. FIG. 8A sets forth the heavy chain variable regions of HC-84.1 (SEQ ID NO:55); HC-84.20 (SEQ ID NO:56); HC-84.21 (SEQ ID NO:57); HC-84.22 (SEQ ID NO:58); HC-23 (SEQ ID NO:59); HC-84.24 (SEQ ID NO:60); HC-84.25 (SEQ ID NO:61); HC-84.26 (SEQ ID NO:62) and HC-84.27 (SEQ ID NO:63). FIG. 8B sets forth the light chain variable regions of HC-84.1 (SEQ ID NO:64); HC-84.20 (SEQ ID NO:65); HC-84.21 (SEQ ID NO:66); HC-84.22 (SEQ ID NO:67); HC-23 (SEQ ID NO:68); HC-84.24 (SEQ ID NO:69); HC-84.25 (SEQ ID NO:70); HC-84.26 (SEQ ID NO:71) and HC-84.27 (SEQ ID NO:72).

### DETAILED DESCRIPTION OF THE EMBODIMENTS

It is to be understood that the invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the polypeptide” includes reference to one or more polypeptides and equivalents thereof known to those skilled in the art, and so forth.

It is further noted that the claims may be drafted to exclude any element which may be optional. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

By “Flaviviridae virus” or “flavivirus” is meant any virus from the Flaviviridae family, including those viruses that infect humans and non-human animals. The polynucleotide and polypeptides sequences encoding these viruses are well known in the art, and may be found at NCBI's GenBank database, e.g., as Genbank Accession nos. NC_{—}004102, AB031663, D11355, D11168, AJ238800, NC_{—}001809, NC_{—}001437, NC_{—}004355 NC_{—}004119, NC_{—}003996, NC_{—}003690, NC_{—}003687, NC_{—}003675, NC_{—}003676, NC_{—}003218, NC_{—}001563, NC_{—}000943, NC_{—}003679, NC_{—}003678, NC_{—}003677, NC_{—}002657, NC_{—}002032, and NC_{—}001461, the contents of which database entries are incorporated by references herein in their entirety. In general the term “flavivirus” includes any member of the family Flaviviridae, including, but not limited to, Dengue virus, including Dengue virus 1, Dengue virus 2, Dengue virus 3, Dengue virus 4 (see, e.g., GenBank Accession Nos. M23027, M19197, A34774, and M14931); Yellow Fever Virus; West Nile Virus; Japanese Encephalitis Virus; St. Louis Encephalitis Virus; Bovine Viral Diarrhea Virus (BVDV); and Hepatitis C Virus (HCV); and any serotype, strain, genotype, subtype, quasispecies, or isolate of any of the foregoing. Where the flavivirus is HCV, the HCV is any of a number of genotypes, subtypes, or quasispecies, including, e.g., genotype 1, including 1a and 1b, 2, 3, 4, 6, etc. and subtypes (e.g., 2a, 2b, 3a, 4a, 4c, etc.), and quasispecies.

The terms “hepatitis C virus,” “HCV,” “non-A non-B hepatitis,” or “NANBH” are used interchangeably herein, and include any “genotype” or “subgenotype” (also termed “subtype”) of the virion, or portion thereof (e.g., a portion of the E2 protein of genotype 1a of HCV), that is encoded by the RNA of hepatitis C virus or that occurs by natural allelic variation. The HCV genome comprises a 5′-untranslated region that is followed by an open reading frame (ORF) that codes for about 3,010 amino acids. The ORF runs from nucleotide base pair 342 to 8,955 followed by another untranslated region at the 3′ end. The amino acids are subdivided into ten proteins in the order from 5′ to 3′ as follows: C; EI; E2; NSI; NS2; NS3; NS4 (a and b); and NS5 (a and b). These proteins are formed from the cleavage of the larger polyprotein by both host and viral proteases. The C, EI, and E2 proteins are structural and the NS1-NS5 proteins are nonstructural proteins. The C region codes for the core nucleocapsid protein. EI and E2 are glycosylated envelope proteins that coat the virus. NS2 may be a zinc metalloproteinase. NS3 is a helicase. NS4a functions as a serine protease cofactor involved in cleavage between NS4b and NS5a. NS5a is a serine phosphoprotein whose function is unknown. The NS5b region has both RNA-dependent RNA polymerase and terminal transferase activity.

There are about six distinct HCV genotypes (e.g., genotypes 1, 2, 3, 4, 5, and 6) that are categorized by variations in the core protein and over 80 subgenotypes which exhibit further variation within each genotype, some of which include: Ia; Ib; Ic; 2a; 2b; 2c; 3a; 3b; 4a; 4b; 4c; 4d; 4e; 5a; and 6a.

As used herein, the terms “neutralizes HCV,” “inhibits HCV,” and “blocks HCV” are used interchangeably to refer to the ability of an antibody of the invention to prevent HCV from infecting a given cell.

The term “effective dose” or “effective dosage” is defined as an amount sufficient to achieve or at least partially achieve the desired effect. The term “therapeutically effective dose” is defined as an amount sufficient to cure or at least partially arrest the disease and its complications in a patient already suffering from the disease. Amounts effective for this use will depend upon the severity of the disorder being treated and the general state of the patient's own immune system.

“Polypeptide” and “protein” as used interchangeably herein, can encompass peptides and oligopeptides. Where “polypeptide” is recited herein to refer to an amino acid sequence of a naturally-occurring protein molecule, “polypeptide” and like terms are not necessarily limited to the amino acid sequence to the complete, native amino acid sequence associated with the recited protein molecule, but instead can encompass biologically active variants or fragments, including polypeptides having substantial sequence similarity or sequence identify relative to the amino acid sequences provided herein. In general, fragments or variants retain a biological activity of the parent polypeptide from which their sequence is derived.

As used herein, “polypeptide” refers to an amino acid sequence of a recombinant or non-recombinant polypeptide having an amino acid sequence of i) a native polypeptide, ii) a biologically active fragment of an polypeptide, or iii) a biologically active variant of an polypeptide. Polypeptides suitable for use can be obtained from any species, e.g., mammalian or non-mammalian (e.g., reptiles, amphibians, avian (e.g., chicken)), particularly mammalian, including human, rodent (e.g., murine or rat), bovine, ovine, porcine, murine, or equine, particularly rat or human, from any source whether natural, synthetic, semi-synthetic or recombinant. In general, polypeptides comprising a sequence of a human polypeptide are of particular interest.

The term “derived from” indicates molecule that is obtained directly from the indicated source (e.g., when a protein directly purified from a cell, the protein is “derived from” the cell) or information is obtained from the source, e.g. nucleotide or amino acid sequence, from which the molecule can be synthesized from materials other than the source of information.

The term “isolated” indicates that the recited material (e.g, polypeptide, nucleic acid, etc.) is substantially separated from, or enriched relative to, other materials with which it occurs in nature (e.g., in a cell). A material (e.g., polypeptide, nucleic acid, etc.) that is isolated constitutes at least about 0.1%, at least about 0.5%, at least about 1% or at least about 5% by weight of the total material of the same type (e.g., total protein, total nucleic acid) in a given sample.

The terms “subject” and “patient” are used interchangeably herein to mean a member or members of any mammalian or non-mammalian species that may have a need for the pharmaceutical methods, compositions and treatments described herein. Subjects and patients thus include, without limitation, primate (including humans), canine, feline, ungulate (e.g., equine, bovine, swine (e.g., pig)), avian, and other subjects. Humans and non-human animals having commercial importance (e.g., livestock and domesticated animals) are of particular interest. As will be evidence from the context in which the term is used, subject and patient refer to a subject or patient susceptible to infection by a Flaviviridae virus, particularly HCV.

“Mammal” means a member or members of any mammalian species, and includes, by way of example, canines; felines; equines; bovines; ovines; rodentia, etc. and primates, particularly humans. Non-human animal models, particularly mammals, e.g. primate, murine, lagomorpha, etc. may be used for experimental investigations.

The term “unit dosage form,” as used herein, refers to physically discrete units suitable as unitary dosages for human and animal subjects, each unit containing a predetermined quantity of compounds calculated in an amount sufficient to produce the desired effect in association with a pharmaceutically acceptable diluent, carrier or vehicle. The specifications for the novel unit dosage forms depend on the particular compound employed and the effect to be achieved, and the pharmacodynamics associated with each compound in the host.

A “pharmaceutically acceptable excipient,” “pharmaceutically acceptable diluent,” “pharmaceutically acceptable carrier,” and “pharmaceutically acceptable adjuvant” means an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, diluent, carrier and adjuvant” as used in the specification and claims includes both one and more than one such excipient, diluent, carrier, and adjuvant.

As used herein, a “pharmaceutical composition” is meant to encompass a composition suitable for administration to a subject, such as a mammal, especially a human. In general a “pharmaceutical composition” is sterile, and is usually free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intracheal and the like.

The term “antibody” is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity. “Antibodies” (Abs) and “immunoglobulins” (Igs) are glycoproteins having the same structural characteristics. While antibodies exhibit binding specificity to a specific antigen, immunoglobulins include both antibodies and other antibody-like molecules which lack antigen specificity. Polypeptides of the latter kind are, for example, produced at low levels by the lymph system and at increased levels by myelomas.

As used in this invention, the term “epitope” means any antigenic determinant on an antigen to which the paratope of an antibody binds. Epitopic determinants usually consist of chemically active surface groupings of molecules such as amino acids or sugar side chains and usually have specific three dimensional structural characteristics, as well as specific charge characteristics.

“Native antibodies and immunoglobulins” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light- and heavy-chain variable domains (Clothia et al., J. Mol. Biol. 186:651 (1985); Novotny and Haber, Proc. Natl. Acad. Sci. U.S.A. 82:4592 (1985)).

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in binding an antibody to an antigen, but exhibit various effector functions, such as participation of the antibody in antibody-dependent cellular toxicity.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)₂ fragment that has two antigen-combining sites and is still capable of cross-linking antigen.

“Fv” is the minimum antibody fragment which contains a complete antigen-recognition and -binding site. In a two-chain Fv species, this region consists of a dimer of one heavy- and one light-chain variable domain in tight, non-covalent association. In a single-chain Fv species (scFv), one heavy- and one light-chain variable domain can be covalently linked by a flexible peptide linker such that the light and heavy chains can associate in a “dimeric” structure analogous to that in a two-chain Fv species. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site. For a review of scFv see Pluckthun, in The Pharmacology of Monoclonal Antibodies, vol. 113, Rosenburg and Moore eds., Springer-Verlag, New York, pp. 269-315 (1994).

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

There are five major classes of immunoglobulins: IgA, IgD, IgE, IgG, and IgM, and several of these can be further divided into subclasses (isotypes), e.g., IgG₁, IgG₂, IgG₃, IgG₄, IgA₁, IgA₂. The heavy-chain constant domains that correspond to the different classes of immunoglobulins are called α, δ, ε, γ, and μ, respectively. The subunit structures and three-dimensional configurations of different classes of immunoglobulins are well known.

The term “human antibody” includes antibodies having variable and constant regions (if present) of human germline immunoglobulin sequences. Human antibodies of the invention can include amino acid residues not encoded by human germline immunoglobulin sequences (e.g., mutations introduced by random or site-specific mutagenesis in vitro or by somatic mutation in vivo). However, the term “human antibody” does not include antibodies in which CDR sequences derived from the germline of another mammalian species, such as a mouse, have been grafted onto human framework sequences (i.e., humanized antibodies).

“Antibody fragment”, and all grammatical variants thereof, as used herein are defined as a portion of an intact antibody comprising the antigen binding site or variable region of the intact antibody, wherein the portion is free of the constant heavy chain domains (i.e. CH2, CH3, and CH4, depending on antibody isotype) of the Fc region of the intact antibody. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules (2) single chain polypeptides containing only one light chain variable domain, or a fragment thereof that contains the three CDRs of the light chain variable domain, without an associated heavy chain moiety and (3) single chain polypeptides containing only one heavy chain variable region, or a fragment thereof containing the three CDRs of the heavy chain variable region, without an associated light chain moiety; and multispecific or multivalent structures formed from antibody fragments. In an antibody fragment comprising one or more heavy chains, the heavy chain(s) can contain any constant domain sequence (e.g. CH1 in the IgG isotype) found in a non-Fc region of an intact antibody, and/or can contain any hinge region sequence found in an intact antibody, and/or can contain a leucine zipper sequence fused to or situated in the hinge region sequence or the constant domain sequence of the heavy chain(s).

Unless specifically indicated to the contrary, the term “conjugate” as described and claimed herein is defined as a heterogeneous molecule formed by the covalent attachment of one or more antibody fragment(s) to one or more polymer molecule(s), wherein the heterogeneous molecule is water soluble, i.e. soluble in physiological fluids such as blood, and wherein the heterogeneous molecule is free of any structured aggregate. A conjugate of interest is PEG. In the context of the foregoing definition, the term “structured aggregate” refers to (1) any aggregate of molecules in aqueous solution having a spheroid or spheroid shell structure, such that the heterogeneous molecule is not in a micelle or other emulsion structure, and is not anchored to a lipid bilayer, vesicle or liposome; and (2) any aggregate of molecules in solid or insolubilized form, such as a chromatography bead matrix, that does not release the heterogeneous molecule into solution upon contact with an aqueous phase. Accordingly, the term “conjugate” as defined herein encompasses the aforementioned heterogeneous molecule in a precipitate, sediment, bioerodible matrix or other solid capable of releasing the heterogeneous molecule into aqueous solution upon hydration of the solid.

The term “monoclonal antibody” (mAb) as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Each mAb is directed against a single determinant on the antigen. In addition to their specificity, the monoclonal antibodies are advantageous in that they can be synthesized by cell culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made in an immortalized B cell or hybridoma thereof, may be made by recombinant DNA methods, including without limitation yeast display.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In some embodiments, the antibody will be purified (1) to greater than 75% by weight of antibody as determined by the Lowry method, and most preferably more than 80%, 90% or 99% by weight, or (2) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The word “label” when used herein refers to a detectable compound or composition which is conjugated directly or indirectly to the antibody. The label may itself be detectable by itself (e.g., radioisotope labels or fluorescent labels) or, in the case of an enzymatic label, may catalyze chemical alteration of a substrate compound or composition which is detectable.

By “solid phase” is meant a non-aqueous matrix to which the antibody of the present invention can adhere. Examples of solid phases encompassed herein include those formed partially or entirely of glass (e.g. controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g. an affinity chromatography column). This term also includes a discontinuous solid phase of discrete particles, such as those described in U.S. Pat. No. 4,275,149.

Other definitions of terms appear throughout the specification.

### Antibody Compositions

Compositions and methods are provided relating to human anti-HCV monoclonal antibodies. The antibodies of the invention bind to and neutralize HCV virus across multiple genotypes. Embodiments of the invention include isolated antibodies and derivatives and fragments thereof, pharmaceutical formulations comprising one or more of the human anti-HCV monoclonal antibodies; cell lines that produce these monoclonal antibodies.

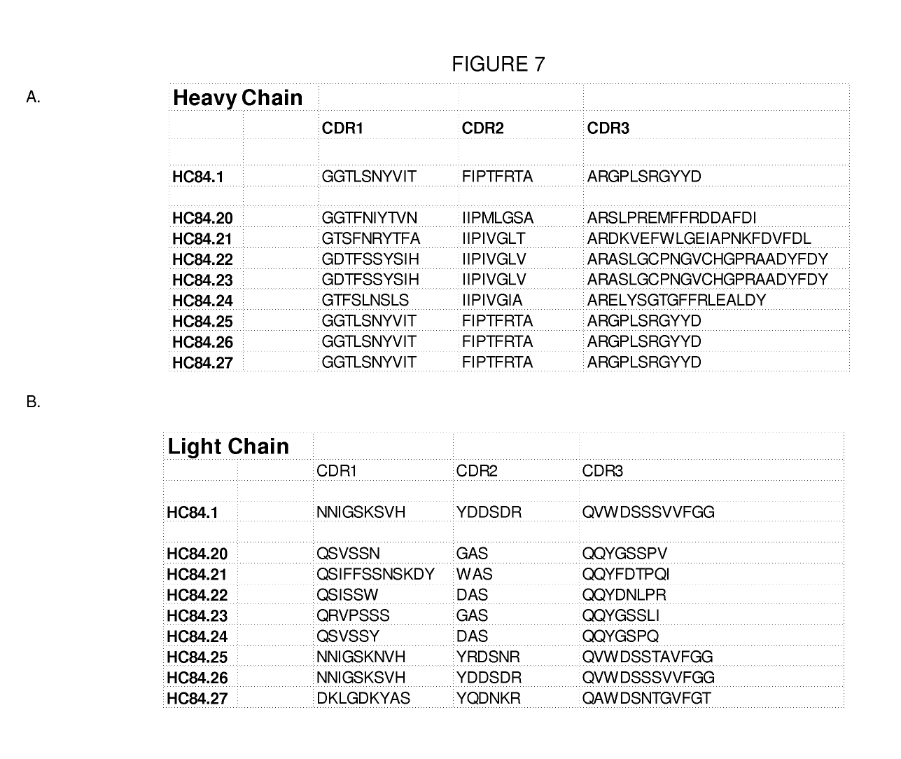

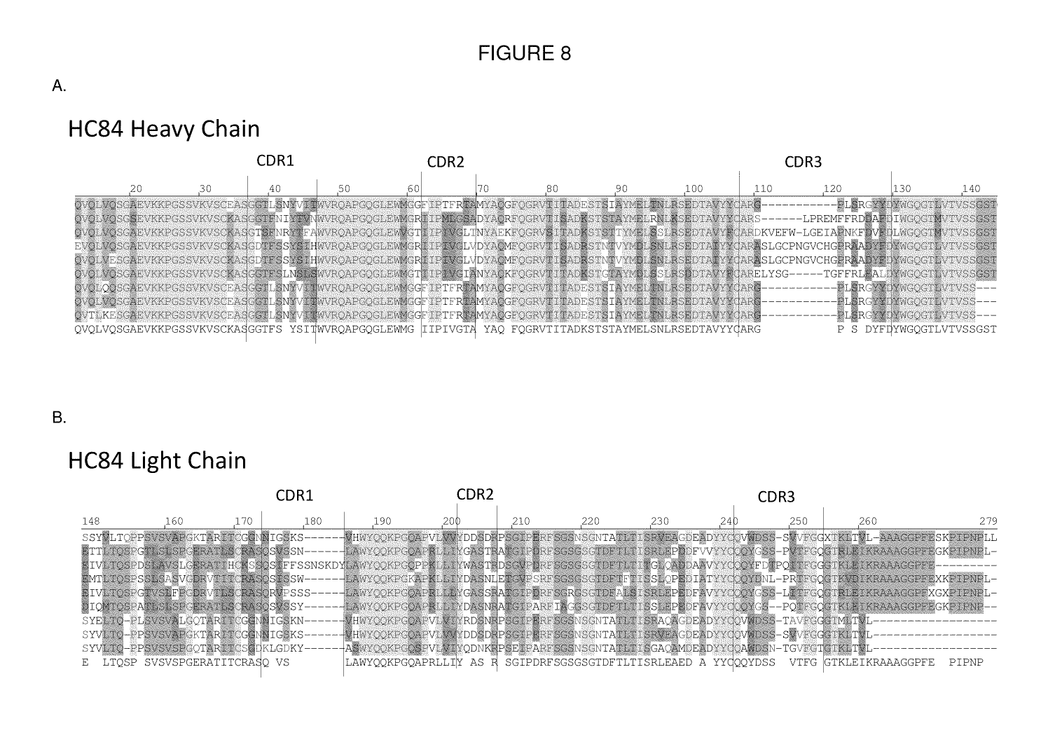

In one aspect, the present invention is directed to combinatorially derived human monoclonal antibodies which are specifically reactive with and neutralize HCV, and cell lines which produce such antibodies. Variable regions of exemplary antibodies are provided, e.g. HC-84.1 (SEQ ID NO:55; SEQ ID NO:64); HC-84.20 (SEQ ID NO:56; SEQ ID NO:65); HC-84.21 (SEQ ID NO:57; SEQ ID NO:66); HC-84.22 (SEQ ID NO:58; SEQ ID NO:67); HC-23 (SEQ ID NO:59; SEQ ID NO:68); HC-84.24 (SEQ ID NO:60; SEQ ID NO:69); HC-84.25 (SEQ ID NO:61; SEQ ID NO:70); HC-84.26 (SEQ ID NO:62; SEQ ID NO:71); and HC-84.27 (SEQ ID NO:63; SEQ ID NO:72). Antibodies of interest include these provided combinations, as well as fusions of the variable regions to appropriate constant regions or fragments of constant regions, e.g. to generate F(ab)′ antibodies. Variable regions of interest include at least one CDR sequence, for example as shown in FIG. 7, where a CDR may be 3, 4, 5, 6, 7, 8, 9, 10, 11, 12 or more amino acids. Alternatively, antibodies of interest include a pair of variable regions as set forth in SEQ ID NO:55, 64; SEQ ID NO:56, 65; SEQ ID NO:57, 66; SEQ ID NO:58, 67; SEQ ID NO:59, 68; SEQ ID NO:60, 69; SEQ ID NO:61, 70; SEQ ID NO:62, 71; and SEQ ID NO:63, 72. In other embodiments, one antibody chain can comprise the CDR sequence set forth in SEQ ID NO:1-3; SEQ ID NO:4-6; SEQ ID NO:7-9; SEQ ID NO:10-12; SEQ ID NO:13-15, SEQ ID NO:16-18; SEQ ID NO:19-21; SEQ ID NO:22-24; SEQ ID NO:25-27. Such an antibody chain may be combined with an antibody chain comprising the CDR sequences set forth in SEQ ID NO:28-30; SEQ ID NO:31-33; SEQ ID NO:34-36; SEQ ID NO:37-39, SEQ ID NO:40-42; SEQ ID NO:43-45; SEQ ID NO:46-48; SEQ ID NO:49-52; SEQ ID NO:53-55.

In some embodiments, a CDR set comprises a heavy and light chain comprising, respectively, the CDR sequences set forth in SEQ ID NO:1-3 and SEQ ID NO:28-30; the CDR sequences set forth in SEQ ID NO:4-6 and SEQ ID NO:31-33; the CDR sequences set forth in SEQ ID NO:7-9 and SEQ ID NO:34-36; the CDR sequences set forth in SEQ ID NO:10-12 and SEQ ID NO:37-39; the CDR sequences set forth in SEQ ID NO:13-15 and SEQ ID NO:40-42; the CDR sequences set forth in SEQ ID NO:16-18 and SEQ ID NO:43-45; the CRD sequences set forth in SEQ ID NO:19-21 and SEQ ID NO:46-48; the CDR sequences set forth in SEQ ID NO:22-24 and SEQ ID NO:49-52; and the CDR sequences set forth in SEQ ID NO:25-27 and SEQ ID NO:53-55.

Antibodies of the invention bind to HCV E2 proteins of different HCV genotypes, including 1A, 2A, 2B, 4A, 5A and 6A. Epitope mapping of antibody binding to HCV E2 protein, shows that the antibodies recognize a conformational epitope, which may include residues 420-429; 441-446; and 613-616. Contact residues may include W420, N428, C429, W437, L441, F442, Y443, K446, Y613 and W616.

One or more residues of a CDR may be altered to modify binding to achieve a more favored on-rate of binding, a more favored off-rate of binding, or both, such that an optimized binding constant is achieved. Affinity maturation techniques are well known in the art and can be used to alter the CDR region(s), followed by screening of the resultant binding molecules for the desired change in binding. In addition to, or instead of, modifications within the CDRs, modifications can also be made within one or more of the framework regions, FRI, FR2, FR3 and FR4, of the heavy and/or the light chain variable regions of a human antibody, so long as these modifications do not eliminate the binding affinity of the human antibody.

In general, the framework regions of human antibodies are usually substantially identical, and more usually, identical to the framework regions of the human germline sequences from which they were derived. Of course, many of the amino acids in the framework region make little or no direct contribution to the specificity or affinity of an antibody. Thus, many individual conservative substitutions of framework residues can be tolerated without appreciable change of the specificity or affinity of the resulting human immunoglobulin. Thus, in one embodiment the variable framework region of the human antibody shares at least 85% sequence identity to a human germline variable framework region sequence or consensus of such sequences. In another embodiment, the variable framework region of the human antibody shares at least 90%, 95%, 96%, 97%, 98% or 99% sequence identity to a human germline variable framework region sequence or consensus of such sequences. In addition to simply binding a linear epitope of an HCV E2 protein, a monoclonal antibody may be selected for its retention of other functional properties of antibodies of the invention, such as binding to multiple genotypes of HVC E2 and/or binding with an ultra-high affinity such as, for example, a K_{D} of 10^{″9} M or lower.

In some embodiments a polypeptide of interest has a contiguous sequence of at least about 10 amino acids as set forth in any one of SEQ ID NO:55-72, at least about 15 amino acids, at least about 20 amino acids, at least about 25 amino acids, at least about 30 amino acids, up to the complete provided variable region. Polypeptides of interest also include variable regions sequences that differ by up to one, up to two, up to 3, up to 4, up to 5, up to 6 or more amino acids as compared to the amino acids sequence set forth in any one of SEQ ID NO:55-72. In other embodiments a polypeptide of interest is at least about 80%, at least about 85%, at least about 90%, at least about 95%, at least about 99% identical to the amino acid sequence set forth in any one of SEQ ID NO:55-72.

The isolation of cells producing monoclonal antibodies of the invention can be accomplished using routine screening techniques, which permit determination of the elementary reaction pattern of the monoclonal antibody of interest. Thus, if a human monoclonal antibody being tested binds to the cognate epitope of one of the provided antibodies, i.e. cross-blocks, and neutralizes HCV, then the human monoclonal antibody being tested and the human monoclonal antibody exemplified herein are equivalent.

It is also possible to determine, without undue experimentation, if a human monoclonal antibody has the same specificity as a human monoclonal antibody of the invention by ascertaining whether the former prevents the latter from binding to or neutralizing HCV, including without limitation an ability to neutralize an HCV virus comprising an N415K mutation in the E2 protein. If the human monoclonal antibody being tested competes with the human monoclonal antibody of the invention, as shown by a decrease in binding by the human monoclonal antibody of the invention, then the two monoclonal antibodies bind to the same, or a closely related, epitope. Still another way to determine whether a human monoclonal antibody has the specificity of a human monoclonal antibody of the invention is to pre-incubate the human monoclonal antibody of the invention with HCV with which it is normally reactive, and then add the human monoclonal antibody being tested to determine if the human monoclonal antibody being tested is inhibited in its ability to bind HCV. If the human monoclonal antibody being tested is inhibited then, in all likelihood, it has the same, or functionally equivalent, epitopic specificity as the monoclonal antibody of the invention. Screening of human monoclonal antibodies of the invention can be also carried out utilizing HCV and determining whether the monoclonal antibody neutralizes HCV.

In addition to Fabs, smaller antibody fragments and epitope-binding peptides having binding specificity for at least one epitope of HCV are also contemplated by the present invention and can also be used to neutralize the virus. For example, single chain antibodies can be constructed according to the method of U.S. Pat. No. 4,946,778 to Ladner et al, which is incorporated herein by reference in its entirety. Single chain antibodies comprise the variable regions of the light and heavy chains joined by a flexible linker moiety. Yet smaller is the antibody fragment known as the single domain antibody, which comprises an isolate VH single domain. Techniques for obtaining a single domain antibody with at least some of the binding specificity of the intact antibody from which they are derived are known in the art. For instance, Ward, et al. in “Binding Activities of a Repertoire of Single Immunoglobulin Variable Domains Secreted from Escherichia coli,” Nature 341: 644-646, disclose a method for screening to obtain an antibody heavy chain variable region (H single domain antibody) with sufficient affinity for its target epitope to bind thereto in isolate form.

### Methods of Use

The invention includes methods of treating an HCV-mediated disease in a subject by administering to the subject an isolated human monoclonal antibody or antigen binding portion thereof as described herein (i.e., that specifically binds to HCV) in an amount effective to inhibit HCV disease, e.g., HCV-mediated symptoms or morbidity. Such diseases may include various liver conditions associated with HCV infection. Treatment of patients before, during and/or after liver transplant. Treatment may include the use of the monoclonal antibodies of the invention as a single agent, or as an agent in combination with additional antiviral agents, including drugs, additional antibodies, vaccines, and the like.

Subjects suspected of having an HCV infection can be screened prior to therapy. Further, subjects receiving therapy may be tested in order to assay the activity and efficacy of the treatment. Significant improvements in one or more parameters is indicative of efficacy. It is well within the skill of the ordinary healthcare worker (e.g., clinician) to adjust dosage regimen and dose amounts to provide for optimal benefit to the patient according to a variety of factors (e.g., patient-dependent factors such as the severity of the disease and the like, the compound administered, and the like). For example, HCV infection in an individual can be detected and/or monitored by the presence of HCV RNA in blood, and/or having anti-HCV antibody in their serum. Other clinical signs and symptoms that can be useful in diagnosis and/or monitoring of therapy include assessment of liver function and assessment of liver fibrosis (e.g., which may accompany chronic viral infection).

Subjects for whom the therapy described herein can be administered include naïve individuals (e.g., individuals who are diagnosed with HCV infection, but who have not been previously treated for HCV) and individuals who have failed prior treatment for HCV (“treatment failure” patients). Previous HCV therapy includes, for example, treatment with IFN-α monotherapy (e.g., IFN-α and/or PEGylated IFN-α) or IFN-α combination therapy, where the combination therapy may include administration of IFN-α and an antiviral agent such as ribavirin. Treatment failure patients include non-responders (i.e., individuals in whom the HCV titer was not significantly or sufficiently reduced by a previous treatment for HCV to provide a clinically significant response, e.g., a previous IFN-α monotherapy, a previous IFN-α and ribavirin combination therapy, or a previous pegylated IFN-α and ribavirin combination therapy); and relapsers (i.e., individuals who were previously treated for HCV (e.g., who received a previous IFN-α monotherapy, a previous IFN-α and ribavirin combination therapy, or a previous pegylated IFN-α and ribavirin combination therapy), in whom the HCV titer decreased to provide a clinically significant response, but in whom the decreased HCV titer was not maintained due to a subsequent increase in HCV titer).

Other subjects for whom the therapy disclosed herein is of interest include subject who are “difficult to treat” subjects due to the nature of the HCV infection. “Difficult to treat” subjects are those who 1) have high-titer HCV infection, which is normally defined as an HCV titer of at least about 10⁵, at least about 5×10⁵, or at least about 10⁶ or more genome copies of HCV per milliliter of serum, 2) are infected with HCV of a genotype that is recognized in the field as being associated with treatment failure (e.g. HCV genotype 1, subtypes thereof (e.g., 1a, 1b, etc.), and quasispecies thereof or 3) both.

Human monoclonal antibodies or portions thereof (and compositions comprising the antibodies or portions thereof) of the invention can be administered in a variety of suitable fashions, e.g., intravenously (IV), subcutaneously (SC), or, intramuscularly (IM) to the subject. The antibody or antigen-binding portion thereof can be administered alone or in combination with another therapeutic agent, e.g., a second human monoclonal antibody or antigen binding portion thereof. In one example, the second human monoclonal antibody or antigen binding portion thereof specifically binds to a second HCV isolate that differs from the isolate bound to the first antibody. In another example, the antibody is administered together with another agent, for example, an antiviral agent. Antiviral agents includes pegylated interferon α, ribivarin, etc. In another example, the antibody is administered together with a polyclonal gamma-globulin (e.g., human gammaglobulin). In another example, the antibody is administered before, after, or contemporaneously with a HCV vaccine.

The human monoclonal antibodies of the invention can be used in vitro and in vivo to monitor the course of HCV disease therapy. Thus, for example, by measuring the increase or decrease in the number of cells infected with HCV or changes in the concentration of HCV present in the body or in various body fluids, it would be possible to determine whether a particular therapeutic regimen aimed at ameliorating the HCV disease is effective.

The monoclonal antibodies of the invention may be used in vitro in immunoassays in which they can be utilized in liquid phase or bound to a solid phase carrier. In addition, the monoclonal antibodies in these immunoassays can be detectably labeled in various ways. Examples of types of immunoassays which can utilize monoclonal antibodies of the invention are competitive and non-competitive immunoassays in either a direct or indirect format. Examples of such immunoassays are the radioimmunoassay (RIA) and the sandwich (immunometric) assay. Detection of the antigens using the monoclonal antibodies of the invention can be done utilizing immunoassays which are run in either the forward, reverse, or simultaneous modes, including immunohistochemical assays on physiological samples. Those of skill in the art will know, or can readily discern, other immunoassay formats without undue experimentation.

The monoclonal antibodies of the invention can be bound to many different carriers and used to detect the presence of HCV. Examples of well-known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the invention. Those skilled in the art will know of other suitable carriers for binding monoclonal antibodies, or will be able to ascertain such, using routine experimentation.

There are many different labels and methods of labeling known to those of ordinary skill in the art. Examples of the types of labels which can be used in the present invention include enzymes, radioisotopes, fluorescent compounds, colloidal metals, chemiluminescent compounds, and bio-luminescent compounds. Those of ordinary skill in the art will know of other suitable labels for binding to the monoclonal antibodies of the invention, or will be able to ascertain such, using routine experimentation. Furthermore, the binding of these labels to the monoclonal antibodies of the invention can be done using standard techniques common to those of ordinary skill in the art.

For purposes of the invention, HCV may be detected by the monoclonal antibodies of the invention when present in biological fluids and tissues. Any sample containing a detectable amount of HCV can be used. A sample can be a liquid such as urine, saliva, cerebrospinal fluid, blood, serum and the like, or a solid or semi-solid such as tissues, feces, and the like, or, alternatively, a solid tissue such as those commonly used in histological diagnosis.

Another labeling technique which may result in greater sensitivity consists of coupling the antibodies to low molecular weight haptens. These haptens can then be specifically detected by means of a second reaction. For example, it is common to use haptens such as biotin, which reacts with avidin, or dinitrophenol, pyridoxal, or fluorescein, which can react with specific anti-hapten antibodies.

As a matter of convenience, the antibody of the present invention can be provided in a kit, i.e., a packaged combination of reagents in predetermined amounts with instructions for performing the diagnostic assay. Where the antibody is labeled with an enzyme, the kit will include substrates and cofactors required by the enzyme (e.g., a substrate precursor which provides the detectable chromophore or fluorophore). In addition, other additives may be included such as stabilizers, buffers (e.g., a block buffer or lysis buffer) and the like. The relative amounts of the various reagents may be varied widely to provide for concentrations in solution of the reagents which substantially optimize the sensitivity of the assay. Particularly, the reagents may be provided as dry powders, usually lyophilized, including excipients which on dissolution will provide a reagent solution having the appropriate concentration.

### Polynucleotides

The invention also provides isolated nucleic acids encoding the human anti-HCV antibodies, vectors and host cells comprising the nucleic acid, and recombinant techniques for the production of the antibody. Exemplary polynucleotides encode the heavy or light chain variable region sequences set forth herein, e.g. SEQ ID NO:55-72.

Nucleic acids of interest may be at least about 80% identical to a sequence that encodes SEQ ID NO:55-72, at least about 85%, at least about 90%, at least about 95%, at least about 99%, or identical. In some embodiments a contiguous nucleotide sequence is at least about 20 nt., at least about 25 nt, at least about 50 nt., at least about 75 nt, at least about 100 nt, and up to the complete coding sequence may be used. Such contiguous sequences may encode a CDR sequence, for example as set forth in SEQ ID NO:1-54, or may encode a complete variable region. As is known in the art, a variable region sequence may be fused to any appropriate constant region sequence.

For recombinant production of the antibody, the nucleic acid encoding it is inserted into a replicable vector for further cloning (amplification of the DNA) or for expression. DNA encoding the monoclonal antibody is readily isolated and sequenced using conventional procedures (e.g., by using oligonucleotide probes that are capable of binding specifically to genes encoding the heavy and light chains of the antibody). Many vectors are available. The vector components generally include, but are not limited to, one or more of the following: a signal sequence, an origin of replication, one or more marker genes, an enhancer element, a promoter, and a transcription termination sequence.

The anti-HCV antibody of this invention may be produced recombinantly not only directly, but also as a fusion polypeptide with a heterologous or homologous polypeptide, which include a signal sequence or other polypeptide having a specific cleavage site at the N-terminus of the mature protein or polypeptide, an immunoglobulin constant region sequence, and the like. A heterologous signal sequence selected preferably may be one that is recognized and processed (i.e., cleaved by a signal peptidase) by the host cell. For prokaryotic host cells that do not recognize and process the native antibody signal sequence, the signal sequence is substituted by a prokaryotic signal sequence selected.

An “isolated” nucleic acid molecule is a nucleic acid molecule that is identified and separated from at least one contaminant nucleic acid molecule with which it is ordinarily associated in the natural source of the antibody nucleic acid. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes a nucleic acid molecule contained in cells that ordinarily express the antibody where, for example, the nucleic acid molecule is in a chromosomal location different from that of natural cells.

The expression “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. Where distinct designations are intended, it will be clear from the context.

Suitable host cells for cloning or expressing the DNA are the prokaryote, yeast, or higher eukaryote cells. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/−DHFR(CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci. 383:44-68 (1.982)); MRC 5 cells; FS4 cells; and a human hepatoma line (Hep G2).

Host cells are transformed with the above-described expression or cloning vectors for anti-HCV antibody production and cultured in conventional nutrient media modified as appropriate for inducing promoters, selecting transformants, or amplifying the genes encoding the desired sequences.

In some embodiments of the invention, the provided human antibody variable regions and/or CDR regions are used in a screening method to select for antibodies optimized for affinity, specificity, and the like. In such screening methods, random or directed mutagenesis is utilized to generate changes in the amino acid structure of the variable region or regions, where such variable regions will initially comprise one or more of the provided CDR sequences, e.g. a framework variable region comprising CDR1, CDR2, CDR3 from the heavy and light chain sequences provided in SEQ ID NO:55-72.

These mutated variable region sequences, which are optionally combined with a second variable region sequence, i.e. V_{H}VL, with constant regions, as a fusion protein to provide for display, etc., as known in the art. Methods for selection of antibodies with optimized specificity, affinity, etc., are known and practiced in the art, e.g. including methods described by Presta (2006) Adv Drug Deliv Rev. 58(5-6):640-56; Levin and Weiss (2006) Mol. Biosyst. 2(1):49-57; Rothe et al. (2006) Expert Opin Biol Ther. 6(2):177-87; Ladner et al. (2001) Curr Opin Biotechnol. 12(4):406-10; Amstutz et al. (2001) Curr Opin Biotechnol. 12(4):400-5; Nakamura and Takeo (1998) J Chromatogr B Biomed Sci Appl. 715(1):125-36 each herein specifically incorporated by reference for teaching methods of mutagenesis selection. Such methods are exemplified by Wu et al. (2005) J. Mol. Biol. (2005) 350, 126-144.

Such screening methods may involve mutagenizing a variable region sequence comprising one or more CDR sequences set forth herein; expressing the mutagenized sequence to provide a polypeptide product; contacting the polypeptide with an HCV antigen; identifying those polypeptide having the desired antigen affinity or specificity.

The antibody composition prepared from the cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the antibody. Protein A can be used to purify antibodies that are based on human γ1, γ2, or γ4 heavy chains (Lindmark et al., J. Immunol. Meth. 62:1-13 (1983)). Protein G is recommended for human γ3 (Guss et al., EMBO J. 5:15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH₃ domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25M salt).

### Formulations

The antibody formulations of the present invention may be used to treat the various HCV associated diseases as described herein. In some embodiments, the recipient is at a high risk of infection.

The antibody formulation is administered by any suitable means, including parenteral, subcutaneous, intraperitoneal, intrapulmonary, and intranasal. Parenteral infusions include intramuscular, intravenous, intraarterial, intraperitoneal, or subcutaneous administration. In addition, the antibody formulation is suitably administered by pulse infusion, particularly with declining doses of the antibody.

For the prevention or treatment of disease, the appropriate dosage of antibody will depend on the type of disease to be treated, the severity and course of the disease, whether the antibody is administered for preventive purposes, previous therapy, the patient's clinical history and response to the antibody, and the discretion of the attending physician. The antibody is suitably administered to the patient at one time or over a series of treatments.

In another embodiment of the invention, an article of manufacture containing materials useful for the treatment of the disorders described above is provided. The article of manufacture comprises a container and a label. Suitable containers include, for example, bottles, vials, syringes, and test tubes. The containers may be formed from a variety of materials such as glass or plastic. The container holds a composition which is effective for treating the condition and may have a sterile access port (for example the container may be an intravenous solution bag or a vial having a stopper pierceable by a hypodermic injection needle). The active agent in the composition is one or more antibodies in a formulation of the invention as described above. The label on, or associated with, the container indicates that the composition is used for treating the condition of choice. The article of manufacture may further comprise a second container comprising a pharmaceutically-acceptable buffer, such as phosphate-buffered saline, Ringers solution and dextrose solution. It may further include other materials desirable from a commercial and user standpoint, including other buffers, diluents, filters, needles, syringes, and package inserts with instructions for use.

Therapeutic formulations comprising one or more antibodies of the invention are prepared for storage by mixing the antibody having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. The antibody composition will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the antibody to be administered will be governed by such considerations, and is the minimum amount necessary to reduce virus titer in an infected individual.

The therapeutic dose may be at least about 0.01 μg/kg body weight, at least about 0.05 μg/kg body weight; at least about 0.1 μg/kg body weight, at least about 0.5 μg/kg body weight, at least about 1 μg/kg body weight, at least about 2.5 μg/kg body weight, at least about 5 μg/kg body weight, and not more than about 100 μg/kg body weight. It will be understood by one of skill in the art that such guidelines will be adjusted for the molecular weight of the active agent, e.g. in the use of antibody fragments, or in the use of antibody conjugates. The dosage may also be varied for localized administration, or for systemic administration, e.g. i.m., i.p., i.v., and the like.

The antibody need not be, but is optionally formulated with one or more agents currently used to prevent or treat HCV infection. These are generally used in the same dosages and with administration routes as used hereinbefore or about from 1 to 99% of the heretofore employed dosages.

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). Formulations to be used for in vivo administration must be sterile. This is readily accomplished by filtration through sterile filtration membranes.

The formulation herein may also contain more than one active compound as necessary for the particular indication being treated, preferably those with complementary activities that do not adversely affect each other. For example, it may be desirable to further provide an antiviral agent. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

A pharmaceutically effective dose is that dose required to prevent, inhibit the occurrence, or treat (alleviate a symptom at least to some extent) of a disease state, e.g. to reduce virus titer in an infected individual. The pharmaceutically effective dose depends on the type of disease, the composition used, the route of administration, the type of subject being treated, subject-dependent characteristics under consideration, concurrent medication, and other factors that those skilled in the medical arts will recognize. Generally, an amount between 0.1 mg/kg and 100 mg/kg body weight/day of active ingredients is administered.

Formulations and methods of delivery of agents to the liver are known in the art, see, e.g., Wen et al., 2004, World J. Gastroenterol. 10:244-9; Murao et al., 2002, Pharm. Res. 19:1808-14; Liu et al., 2003, Gene Ther. 10:180-7; Hong et al., 2003, J. Pharm. Pharmacol. 54; 51-8; Herrmann et al., 2004, Arch. Virol. 149:1611-7; and Matsuno et al., 2003, Gene. Ther. 10:1559-66.

Formulations and methods of delivery of agents to the skin or mucosa are known in the art. Such delivery systems include, e.g., aqueous and nonaqueous gels, creams, multiple emulsions, microemulsions, liposomes, ointments, aqueous and nonaqueous solutions, lotions, patches, suppositories, and tablets, and can contain excipients such as solubilizers, permeation enhancers (e.g., fatty acids, fatty acid esters, fatty alcohols and amino acids), and hydrophilic polymers (e.g., polycarbophil and polyvinylpyrolidone).

Oral administration can be accomplished using pharmaceutical compositions containing an agent of interest formulated as tablets, lozenges, aqueous or oily suspensions, dispersible powders or granules, emulsion, hard or soft capsules, or syrups or elixirs. Such oral compositions can contain one or more such sweetening agents, flavoring agents, coloring agents or preservative agents in order to provide pharmaceutically elegant and palatable preparations. Tablets, which can be coated or uncoated, can be formulated to contain the active ingredient in admixture with non-toxic pharmaceutically acceptable excipients, e.g., inert diluents; such as calcium carbonate, sodium carbonate, lactose, calcium phosphate or sodium phosphate; granulating and disintegrating agents, for example, corn starch, or alginic acid; binding agents, e.g., starch, gelatin or acacia; and lubricating agents, for example magnesium stearate, stearic acid or talc. Where a coating is used, the coating delay disintegration and absorption in the gastrointestinal tract and thereby provide a sustained action over a longer period.

Where the formulation is an aqueous suspension, such can contain the active agent in a mixture with a suitable excipient(s). Such excipients can be, as appropriate, suspending agents (e.g., sodium carboxymethylcellulose, methylcellulose, hydropropyl-methylcellulose, sodium alginate, polyvinylpyrrolidone, gum tragacanth and gum acacia); dispersing or wetting agents; preservatives; coloring agents; and/or flavoring agents.

Suppositories, e.g., for rectal administration of agents, can be prepared by mixing the agent with a suitable non-irritating excipient that is solid at ordinary temperatures but liquid at the rectal temperature and will therefore melt in the rectum to release the drug. Such materials include cocoa butter and polyethylene glycols.

Dosage levels can be readily determined by the ordinarily skilled clinician, and can be modified as required, e.g., as required to modify a subject's response to therapy. In general dosage levels are on the order of from about 0.1 mg to about 140 mg per kilogram of body weight per day. The amount of active ingredient that can be combined with the carrier materials to produce a single dosage form varies depending upon the host treated and the particular mode of administration. Dosage unit forms generally contain between from about 1 mg to about 500 mg of an active ingredient.

### Peptide Vaccine Compositions

The application discloses herein a method of inducing an immune response against a peptide corresponding to an epitope recognized by an HC-84 antibody, including peptides comprising residues 420, 428, 429, 437, 441, 442, 443, 446, 613 and 616 of HCV E2 protein, where the epitope is of sufficient length to provide for binding specificity substantially similar to the specificity of binding to the native protein, e.g. a peptide of at least 20 amino acids, at least 30 amino acids, at least 40 amino acids, at least 50 amino acids, at least 100 amino acids, at least 150 amino acids, at least 200 amino acids up to the full length of the E2 protein, where the peptide may be a contiguous or non-contiguous sequence of an HCV E2 protein.

In some embodiments, all or a portion of the HCV E2 protein is provided as an antigen, where specific highly immunodominant residues are masked, so as to allow for the generation of an immune response to residues that are less immunodominant, but which are essential for virus function and therefore are less likely to be altered in virus escape mutation and selection. These antigens find use in screening assays, generation of monoclonal antibodies, and in vaccines. Residues of interest for masking include Y632 and D535, which can be masked by substituting the native amino acid with alanine, serine, or other small uncharged amino acid. Peptides for immunization may be conjugated to a carrier molecule prior to administration to a subject.

Peptides can be produced using techniques well known in the art. Such techniques include chemical and biochemical synthesis. Examples of techniques for chemical synthesis of peptides are provided in Vincent, in Peptide and Protein Drug Delivery, New York, N.Y., Dekker, 1990. Examples of techniques for biochemical synthesis involving the introduction of a nucleic acid into a cell and expression of nucleic acids are provided in Ausubel, Current Protocols in Molecular Biology, John Wiley, and Sambrook, et al in Molecular Cloning, A Laboratory Manual, Cold Spring Harbor Laboratory Press, 1989.

In the methods disclosed herein, an immunologically effective amount of one or more immunogenic polypeptides, which may be conjugated to a suitable carrier molecule, is administered to a patient by successive, spaced administrations of a vaccine, in a manner effective to result in an improvement in the patient's condition.

In an exemplary embodiment, immunogenic polypeptides are coupled to one of a number of carrier molecules, known to those of skill in the art. A carrier protein must be of sufficient size for the immune system of the subject to which it is administered to recognize its foreign nature and develop antibodies to it.

In some cases the carrier molecule is directly coupled to the immunogenic peptide. In other cases, there is a linker molecule inserted between the carrier molecule and the immunogenic peptide. For example, the coupling reaction may require a free sulfhydryl group on the peptide. In such cases, an N-terminal cysteine residue is added to the peptide when the peptide is synthesized. In an exemplary embodiment, traditional succinimide chemistry is used to link the peptide to a carrier protein. Methods for preparing such peptide:carrier protein conjugates are generally known to those of skill in the art and reagents for such methods are commercially available (e.g., from Sigma Chemical Co.). Generally about 5-30 peptide molecules are conjugated per molecule of carrier protein.

Exemplary carrier molecules include proteins such as keyhole limpet hemocyanin (KLH), bovine serum albumin (BSA), flagellin, influenza subunit proteins, tetanus toxoid (TT), diphtheria toxoid (DT), cholera toxoid (CT), a variety of bacterial heat shock proteins, glutathione reductase (GST), or natural proteins such as thyroglobulin, and the like. One of skill in the art can readily select an appropriate carrier molecule. In some cases, the carrier molecule is a non-protein, such as Ficoll 70 or Ficoll 400 (a synthetic copolymer of sucrose and epichlorohydrin), a polyglucose such as Dextran T 70.

Another category of carrier proteins is represented by virus capsid proteins that have the capability to self-assemble into virus-like particles (VLPs). Examples of VLPs used as peptide carriers are hepatitis B virus surface antigen and core antigen, hepatitis E virus particles, polyoma virus, and bovine papilloma virus.

A peptide vaccine composition may comprise single or multiple copies of the same or different immunogenic peptide, coupled to a selected carrier molecule. In one aspect of this embodiment, the peptide vaccine composition may contain different immunogenic peptides with or without flanking sequences, combined sequentially into a polypeptide and coupled to the same carrier. Alternatively, immunogenic peptides, may be coupled individually as peptides to the same or a different carrier, and the resulting immunogenic peptide-carrier conjugates blended together to form a single composition, or administered individually at the same or different times.

In general, peptide vaccine compositions are administered with a vehicle. The purpose of the vehicle is to emulsify the vaccine preparation. Numerous vehicles are known to those of skill in the art, and any vehicle which functions as an effective emulsifying agent finds utility in the present invention. To further increase the magnitude of the immune response resulting from administration of the vaccine, an immunological adjuvant may be included in the vaccine formulation. Exemplary adjuvants known to those of skill in the art include water/oil emulsions, non-ionic copolymer adjuvants, e.g., CRL 1005 (Optivax; Vaxcel Inc., Norcross, Ga.), aluminum phosphate, aluminum hydroxide, aqueous suspensions of aluminum and magnesium hydroxides, bacterial endotoxins, polynucleotides, polyelectrolytes, lipophilic adjuvants and synthetic muramyl dipeptide (norMDP) analogs.

Suitable pharmaceutically acceptable carriers for use in an immunogenic proteinaceous composition of the invention are well known to those of skill in the art. Such carriers include, for example, phosphate buffered saline, or any physiologically compatible medium, suitable for introducing the vaccine into a subject.

Numerous drug delivery mechanisms known to those of skill in the art may be employed to administer the immunogenic peptides of the invention. Controlled release preparations may be achieved by the use of polymers to complex or absorb the peptides or antibodies. Controlled delivery may accomplished using macromolecules such as, polyesters, polyamino acids, polyvinyl pyrrolidone, ethylenevinylacetate, methylcellulose, carboxymethylcellulose, or protamine sulfate, the concentration of which can alter the rate of release of the peptide vaccine.

In some cases, the peptides may be incorporated into polymeric particles composed of e.g., polyesters, polyamino acids, hydrogels, polylactic acid, or ethylene vinylacetate copolymers. Alternatively, the peptide vaccine is entrapped in microcapsules, liposomes, albumin microspheres, microemulsions, nanoparticles, nanocapsules, or macroemulsions, using methods generally known to those of skill in the art.

The vaccine of the present invention can be administered to patient by different routes such as intravenous, intraperitoneal, subcutaneous, intramuscular, or orally. A preferred route is intramuscular or oral. Suitable dosing regimens are preferably determined taking into account factors well known in the art including age, weight, sex and medical condition of the subject; the route of administration; the desired effect; and the particular conjugate employed (e.g., the peptide, the peptide loading on the carrier, etc.). The vaccine can be used in multi-dose vaccination formats.

It is expected that a dose would consist of the range of to 1.0 mg total protein. In an embodiment of the present invention the range is 0.1 mg to 1.0 mg. However, one may prefer to adjust dosage based on the amount of peptide delivered. In either case these ranges are guidelines. More precise dosages should be determined by assessing the immunogenicity of the conjugate produced so that an immunologically effective dose is delivered. An immunologically effective dose is one that stimulates the immune system of the patient to establish a level immunological memory sufficient to provide long term protection against disease caused by infection with HCV. The conjugate is preferably formulated with an adjuvant.

The timing of doses depend upon factors well known in the art. After the initial administration one or more booster doses may subsequently be administered to maintain antibody titers. An example of a dosing regime would be a dose on day 1, a second dose at or 2 months, a third dose at either 4, 6 or 12 months, and additional booster doses at distant times as needed.

In one aspect, the invention provides a means for classifying the immune response to peptide vaccine, e.g., 9 to 15 weeks after administration of the vaccine; by measuring the level of antibodies against the immunogenic peptide of the vaccine.

The invention now being fully described, it will be apparent to one of ordinary skill in the art that various changes and modifications can be made without departing from the spirit or scope of the invention.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

### Materials and Methods

Cell Culture, Antibodies, Virus and Reagents.

HEK-293T cells were obtained from the ATCC. Huh7.5 cells (generously provided by Dr. C. Rice, Rockefeller University) were grown in Dulbecco's modified minimal essential medium (Invitrogen, Carlsbad, Calif.) supplemented with 10% fetal calf serum (Sigma-Aldrich Co., St. Louis, Mo.) and 2 mM glutamine.

Yeast Strain

EBY-100 (GAL1-AGA1:URA3 ura3-52 trp1 leu2Δ1 his3Δ200 pep4::HIS2 prb1Δ1.6R can1 GAL) was maintained in YPD broth (Difco). HMAbs CBH-5, HC-1, HC-11, CBH-4G and H-111 against HCV E1 and E2 were generated as described previously. MAb against HCV NS3 protein was generously provided by Dr. G. Luo (University of Kentucky). MAb against human CD81 (clone JS-81) was purchased from BD Bioscience (San Jose, Calif.). MAb against V-5 tag was purchased from Invitrogen (Carlsbad, Calif.). The detection MAbs used in Fluorescence-activated cell sorting (FACS): Phycoerythrin (PE)-labeled donkey-anti-human IgG (Fcγ specific) FITC-labeled goat-anti-mouse IgG (Fcγ specific) or Allophycocyanin (APC)-conjugated donkey-anti-human IgG (Fcγ specific) were all purchased from Jackson ImmunoResearch Laboratories (West Grove, Pa.). JFH-12a cell culture infectious virus (HCVcc) was generously provided by Dr. T. Wakita (National Institute of Infectious Diseases, Japan). 1a HCVcc was generously provided by Dr. S. Lemon (University of North Caroline). A molecular clone encoding the CD81 large extracellular loop fused to glutathione S-transferase was generously provided by Dr. S. Levy (Stanford University) and affinity purified over a GSTrap FF affinity column according to the manufacturer's instructions (GE Healthcare Bio-Sciences AB, Uppsala, Sweden). Recombinant HCV E2 derived from different genotypes were generously provided by Dr. J. K. Ball (University of Nottingham). The yeast display vector pYD3-A2 was kindly provided by Dr. James. D Marks (UCSF). Full length IgG1 expression vectors was kindly provided by Dr. P. Wilson (University of Chicago). Biotinylated peptides were synthesized using a C-terminal biotin residue with a gly-ser-gly linker (American Peptide, Sunnyvale, Calif.).

Antigen Preparation.

To test the possibility that rational designed antigen would lead to desired antibodies from immune library screening, two different sets of HCV E2 antigens were constructed: E2_{Y632A} and E2_{D535A} as domain A (non-neutralizing) and B (neutralizing through blocking E2-CD81 interaction), based on earlier epitope mapping studies. This enabled us to deplete positive binders from domain A and B antibodies and increase the yield of non-domain B, but neutralizing antibodies. HCV E2 regions (aa 384 to 661 or 384-718) were cloned into the expression vector pSec in frame with the IgK signal peptide sequence and fused with a myc and six-histidine tag at the carboxyl terminus. The constructs carry respectively the H77c 1a E2 sequence or a genotype 1b E2 coding sequence (GenBank accession nos. AF009606 and AF348705). The alanine substitutions were then introduced at residues 632 or 535 using a QuikChange II site-directed mutagenesis kit (Agilent, La Jolla, Calif.) in accordance with the manufacturer's instructions and mutations were confirmed by DNA sequence analysis (Sequetech, Mountain View, Calif.). The constructs were transfected into HEK293T cells via calcium-phosphate method and the supernatant was harvested after 5 days. E2 protein was affinity purified over His-trap columns. The final product was greater than 90% pure as judged by sodium dodecyl sulfatepolyacrylamide gel electrophoresis (SDS-PAGE) analysis. The conservation of E2 integrity and antigenicity were confirmed using a panel of Hams antibodies representing different domains by ELISA.

Generation of Immune Yeast Antibody Libraries.

An immune library was constructed from peripheral blood donated by a HCV 2b chronic infected individual with laboratory confirmed positive HCV infection. Total RNA, prepared from serum sample, was converted into cDNA using random hexamers. The cDNA products were used in primary PCR reactions to amplify the gamma HC, kappa LC and lambda LC using the primers as described elsewhere with following modifications: for V_{H} primers, the sequences (5′-GT GGT GGT GGT TCT GCT AGC GGG GCC ATG GCC-3′ underlined is a NcoI site), (5′-ACC TCC GGA GCC ACC TCC GCC TGA ACC GCC TCC ACC TGT CGA CCC-3′ underlined is a SalI site) were added to both the 5′ end of the forward and reverse primers respectively. For V_{L} primers, the sequences (5′-C GGT TCA GGC GGA GGT GGC TCC GGA GGT GGC GGA TCG-3′ underlined is a BspE1 site), and (5′-GG GAT AGG CTT ACC TTC GAA GGG CCC GCC TGC GGC CGC-3′ underlined is a NotI site) were added to all forward Vλ and Vκ primers and all reverse Vλ and Vκ primers, respectively. In addition, yeast display vector pYD2.A2 displaying A2-scFv was created with modified pYD2 vector, which comprises a [(Gly4-Ser)3] linker region carrying SalI and BspEI restriction sites and NcoI and NotI restriction sites flanking the inserted scFv. PCR-amplified heavy chain genes were pooled and cloned into vector pYD2.A2 using NcoI and SalI, yielding an heavy chain library of 5.0×10⁶ clones and the library was further digested with BspE1 and NotI for gap repair with light chain. In parallel, PCR-amplified light chain genes were pooled and ligated into vector pYD2.A2 using BspE1 and NotI, yielding an light chain library of 5.0×10⁶ clones. The V_{L} genes from the light chain library were re-amplified with primers HuJHF and Gap3 (HuJHF: 5′-ACC GTC TCC TCA GGG TCG ACA-3′, Gap3: 5′-GAG ACC GAG GAG AGG GTT AGG-3′), the resulting repertoire (10 μg) was then directly cloned into 50 μg BspEI and NotI pre-digested pYD2.A2.VH library through gap repair transformation into Saccharomyces cerevisiae strain EBY100. Library size was determined by plating serial dilutions of the transformation mixture on SD-CAA plates. This result in the yeast surface display immune yeast antibody libraries of 2×10⁷ clones. To validate the libraries, insert frequency and diversity were analyzed using colony PCR, DNA sequencing and SDS-PAGE/Western blot analysis. Inserts of the correct size and their diversity were found in ˜90-100% of tested vectors.

Yeast Display Selection of HCV E2 Glycoprotein Specific scFv.

The yeast libraries were grown in SG-CAA for 48 hours at 18° C. MACS (Miltenyi, Auburn, Calif.) sorting was performed in accordance with the manufacturer's instructions. For the first MACS selection round 2×10⁹ yeast cells were incubated with domain A depletion E2 proteins (E2_{Y632A} aa 384-661 and 384-718) at 4° C. for 20 min before loading onto a pre-treated column containing 25 ul of anti-myc microbeads. The column was then washed, followed by elution of bound yeast cells with 7 ml of SDCAA media and plunger to push the cell out of the column, and then centrifuge 2500 g for 5 mins. The pellet was resuspended in SDCAA and amplified in SD-CAA, followed by induction in SG-CAA. For the second FACS selection round 1×10⁸ MACS output cells (as non-domain A cell population) were enriched by incubation with the same E2 proteins (E2_{Y632A} aa384-661 and 384-718) at 4° C. for 30 min in FACS wash buffer, then washed in cold wash buffer. The cells were then incubated with anti-V5 (1:5000) and HC-33.1 (anti-E2 monoclonal antibody) (10 μg/ml) for 1 hour at 4° C., followed by another incubation with FITC-anti-mouse (1:200) and PE or APC-anti-human IgG (Fcγ specific) for 30 minutes at 4° C. in the dark to probe the cells. The labeled cells were washed and resuspended in FACS wash buffer at concentration of 1×10⁸/ml for sorting by flow cytometry. Selection were performed using a BD Bioscience FACS Vantage Sorter and sort gates were set to collect the desired double positive cells. Collected cells were grown in SD-CAA media and used for the next round of sorting after induction in SG-CAA, as described above. For the third FACS selection round, 1×10⁷ or 5×10⁶ yeast cells were incubated with domain B depletion E2 proteins (E2_{D535A} aa384-661 and 384-718), the sorting was performed as described above for the second FACS selection round. After the final selection round collected cells were plated on SD-CAA plates and grown at 30° C. for ˜2 days. After the final round of sorting, individual clones were picked, induced and stained with E2 proteins, detected by anti-E2 and anti-V5 antibody. Positive clones were sequenced to identify unique antibody sequences using primers PYFD and PYDR (PYDFor: 5′-AGT AAC GTT TGT CAG TAA TTG C-3′; PYDRev: 5′-GTC GAT TTT GTT ACA TCT ACA C-3′). The PCR product was then gel purified and sequenced with primer GAP5 (Gap 5: 5′-TTA AGC TTC TGC AGG CTA GTG-3′).

Production of IgG1 MAbs.

Reformatting of scFv yeast into full-length IgG1 molecules was essentially as described. In brief, VH and VL genes were PCR amplified using primers to restore the human framework and append restriction sites. The resulting fragments were cloned into Igγ, Igκ or Igλ mammalian expression vectors containing the signal peptide and constant region genes. IgG1 was expressed by co-transfection of 293T cell lines and cultured in serum-free medium. The expression levels were measured by titration of IgG/κ or IgG/λ binding by ELISA and the resulting IgG₁ were purified using protein A affinity chromatography. After determine the resulting IgG1 by ELISA the purity and integrity of the HMAbs were analyzed by reducing and nonreducing SDS-PAGE.

HCVcc Neutralization Assay by Focus-Forming Unit Reduction.

Infectious genotype 2a JFH-1 virus employed for neutralization and variant viruses in escaping HC-84 antibodies generation were essentially as described in detail elsewhere. The neutralization activity of HMAbs against different genotypes of HCVcc and neutralization escape HCVcc mutants was evaluated as previously described. Briefly, a virus inoculum (containing 50 FFU) was incubated with serial dilutions of antibodies for 1 hr at 37° C. before inoculation onto Huh-7.5 cells (3.2×10⁴ cells/well) seeded 24 hrs previously into 8-well chamber slides (Nalge Nunc, Rochester, N.Y.). In the case of peptide competition assay, testing antibody HC-84.25 was diluted at concentrations ranging from 0.00005 to 0.1 ug/ml in PBS containing competing peptide at constant concentration of 2 ug/ml. After 3 hrs of incubation at 37° C. in the presence of 5% CO₂, the inoculum was replaced with 400 μl of fresh complete medium followed by incubation for an additional 72 hrs. Infected cells were fixed and examined for NS3 protein expression by immunofluorescence detection of foci as described in viral titration above. The entire well was visualized in approximately 16 non-overlapping fields to obtain the number of foci. Each experiment was performed in triplicates. The antibody concentrations (μg/ml) causing 50% reductions in FFU were determined by linear-regression analysis. The percent neutralization was calculated as the percent reduction in FFU compared with virus incubated with an irrelevant control antibody. All assays were performed in triplicates.

HCV-Pseudotyped Retroviral Particle Production, Infection and Neutralization Assay.

HCVpp were produced as described previously by cotransfection of 293T cells with pNL4-3.Luc.R⁻E⁻ plasmid containing the env-defective HIV proviral genome and an expression plasmid encoding the HCV glycoproteins or mutant E1E2 proteins. Briefly, for the neutralization assay, the virus-containing medium was incubated with each HMAb at various concentrations, or phosphate-buffered saline instead of the antibodies as an infectivity control, plus 4 μg/ml polybrene at 37° C. for 60 minutes. The HCVpp-antibody mixture was transferred to Huh7.5 cells (8×10³ cells/well) preseeded in 96-well plates, and infections were centrifuged at 730×g for 2 hrs at room temperature. After incubation at 37° C. in the presence of 5% CO₂ for 15 hrs, the unbound virus was replaced with fresh complete medium, followed by additional incubation for a total 72 hrs. The neutralizing activity of an antibody was calculated as the percent reduction of luciferase activity compared with an inoculum containing phosphate-buffered saline (PBS). For HCVpp infectivity studies, the virus-containing extracellular medium was normalized for HIV p24 expression by using a QuickTiter lentivirus titer kit (Cell Biolabs, San Diego, Calif.). All assays were performed in triplicates.

Quantitative Enzyme-Linked Immunoassays.

ELISA was performed to measure antibody binding to the wt E1E2 from different genotypes or mutant E2 glycoproteins and to measure E2 binding to CD81, as described previously. Briefly, microtiter plates were prepared by coating each well with 500 ng of GNA and blocking with 2.5% non-fat dry milk and 2.5% normal goat serum. Lysates of cell expressing wt HCV E1E2 from different genotypes, mutant E1E2, denatured E1E2 protein or pelleted virus were captured by GNA on the plate and later bound by a range of 0.01-100 μg/ml of HMAb. E1E2 protein was denatured by incubation with 0.5% sodium dodecyl sulfate and 5 mM dithiothreitol for 15 min at 56° C. The bound HMAb was detected by incubation with alkaline phosphatase-conjugated goat anti-human IgG (Promega; Madison, Wis.), followed by incubation with p-nitrophenyl phosphate for color development. Absorbance was measured at 405 nm and 570 nm.

For CD81 binding analysis, 20% sucrose cushion-pelleted HCVpp were re-suspended in NTE buffer (150 mM NaCl, 1 mM EDTA, 10 mM Tris-HCl, pH 7.4) and captured on GNA-coated plates. Bound antigen was first incubated with purified recombinant fusion proteins containing the large extracellular loop of human CD81 at concentrations from 0.5 to 500 μg/ml. The amount of HCVpp captured in each well was determined by probing the cells with H-111, a HMAb directed against a linear epitope within E1. The bound CD81 was visualized with AP-conjugated mouse anti-human CD81 (Santa Cruz Biotechnologies, Santa Cruz, Calif.). Color development and absorbance measurement were performed, as described above.

Competition for Binding to E2 Glycoprotein Between HC-84 HMAbs and Antibodies from Other Domains.

Antibody competition assay was performed as described previously in detail. Briefly, an ELISA was used to measure the competition between unlabeled and biotinylated antibodies for binding to immobilized 1a E2 glycoprotein. Each biotinylated test antibody at 2 μg/ml with competing HMAb ranging from 0.2 to 50 μg/ml was tested in duplicate in at least two different experiments. To develop a cross-competition matrix for percentage of test antibody bound to E2, the mean signal with biotinylated test antibody to E2 with competing antibody at 20 μg/ml was divided by the signal in the absence of the competing HMAb, followed by multiplying by 100. The HC-84 HMAbs spatial relationships to the existing domains and among themselves was determined based on competition study results and analyzed using the principles of UPGMA (unweighted pair-group method using arithmetic averages).

Inhibition of Binding of E2 Glycoprotein to CD81.

HMAbs at different concentrations were incubated for 20 min at 4° C. with lysates of cell expressing wt HCV E1E2. The mixture was added to microtiter plates pre-coated with anti-GST and captured recombinant fusion proteins containing the large extracellular loop of human CD81 fused to glutathione S-transferase. After 1 hr incubation at 4° C. with gentle agitation, the wells were washed and 5 ug/ml of anti-cmyc was added to the wells, followed by incubation and addition of 100 μl/well of 1/10,000-diluted alkaline phosphatase-conjugated anti-mouse IgG (Promega, Madison, Wis.). After color development the plate was read at 405/570 nm using spectroMax 190. The percentage of binding inhibition was calculated as reduction of E2 binding to CD81 comparing to the value obtained in the absence of antibody. Background signals for binding of E2 to human CD81 were determined from wells coated with murine CD81-LEL; signals obtained with biotinylated CBH-4D and E2 in the presence of competing antibody were compared to signals obtained from CBH-4D and E2 in the absence of competing antibody.

Immunoprecipitation, CD81 Pull-Down Assay and Western Blotting.

For IP E1E2 proteins, IgG1 was coupled to protein A beads (Thermo Fisher Scientific, Rockford, Ill.) and then incubated with lysates of 293T cells transiently producing E1E2 proteins. Lysates were precleared with empty protein A beads. The beads were washed extensively, and bound immunocomplexes were eluted by the addition of Laemmli buffer. The eluates were separated by SDS-PAGE. For CD81 pull down assay, recombinant fusion proteins containing the large extracellular loop of human CD81 fused to glutathione S-transferase were preadsorbed onto glutathione-Sepharose beads (GE Healthcare) and then incubated with lysates of pseudoparticles producing cells. After incubation at 4° C. for 1.5 hours, beads were extensively washed with lysis buffer. Pull down was followed by Western blotting to detect E1 and E2. After separation by SDS-PAGE, proteins were transferred to nitrocellulose membranes (Bio-Rad Laboratories) and incubated with specific antibodies (H111 as anti-E1 and HC-84.1 as anti-E2) followed by sheep anti-human IgG conjugated to peroxidase. The proteins of interest were revealed by enhanced chemiluminescence detection (GE Healthcare) as recommended by the manufacturer.

SPR Analysis of HC84 Antibodies.

IgG binding kinetics were measured using surface plasmon resonance in a BIAcore3000 (PharmaciaBiosensor) and used to calculate the K_{D}. The HC84 antibodies were captured using a human antibody capture kit (GE healthcare, Biacore. BR-1008-39). Approximately 7000 response units (RU) of mouse anti-human antibody were coupled to a CM5 sensor chip by using N-hydroxysuccinimide (NHS) and N-ethyl-N¢-(dimethylaminopropyl)-carbodiimide (EDC). Approximately 150 RU of purified IgG in Hepes-buffered saline (pH 7.4) (HBS) were captured onto the surface. The purified E2 (384-661) or synthetic peptide at concentrations ranging from 89 nM to 1.42 μM was injected for 2 minutes using a flow rate of 30 μl/min. Dissociation of bound antigen in HBS buffer flow was followed for 3 minutes. The surfaces (HC84 antibodies and E2) were regenerated after each cycle using regeneration solution (3M MgCl₂). The association rate constant (Kₒₙ) was determined from a plot of (ln (dR/dt))/t versus concentration. The dissociation rate constant (K_{off}) was determined from the dissociation part of the sensorgram at the highest concentration of E2. K_{D} was calculated as K_{off}/Kₒₙ, using data from two independent experiments.

Site-Directed Mutagenesis.

All mutagenesis was conducted using a QuikChange II site-directed mutagenesis kit (Agilent, La Jolla, Calif.) in accordance with the manufacturer's instructions. A plasmid containing the 2a JFH-1 E1E2-encoding sequence was used as the template for introducing single and double mutations. Alanine substitution mutants of full-length E2 (residues at 834 to 746) or without TM region (residues at 834 to 746) were constructed in plasmids carrying respectively the H77c 1a E1E2 coding sequence or yeast display pYD2 vector (GenBank accession nos. AF009606) to epitope map HC-84 HMAbs. All the mutations were confirmed by DNA sequence analysis (Sequetech, Mountain View, Calif.) for the desired mutation and for exclusion of unexpected residue changes in the full-length E1E2 encoding sequence. The resulting plasmids were transfected into HEK293T cells for transient protein expression using the calcium-phosphate method. The resulting yeast plasmids were carried out as described below. The mutated constructs were designated X#Y, where # is the residue location in H77c, X denotes the single-letter code for the H77c amino acid, and Y denotes the altered amino acid.

Epitope Mapping.

Epitope mapping was performed using alanine substitution mutations of three defined E2 regions (region 1: aa418-446; region 2: 526-536; region 3: 611-617) by ELISA. To fill in the gap and further confirm the ELISA results a yeast display library comprising a full-length E2 (residues at 834 to 716) was screened. Yeast expressing alanine substitution mutants of E2 were cultured and induced as described above, and screened in a 96-wells format by flow cytometry. The library was pre-screened by representative domain A (CBH-4D) and domain C (CBH-7) antibodies to ensure the structural integrity. The corresponding yeast clones that remain binding activities to the reference antibodies was used to further screen for those lost specific binding to individual HC-84 antibodies. In the second round screening the clones that were positive to anti-V5 but lost binding to individual HC-84 antibody were identified, plasmid were extracted, purified and sequenced (Elim Biopharm, Hayward, Calif.).

Generation of Mutated JFH-1 Viruses Escaping Neutralization by HC-84 Antibodies.

Infectious genotype 2a JFH-1 virus employed for the escape mutation studies and variant viruses in escaping HC-84 antibodies generation were essentially as described in detail elsewhere. Generation of mutated JFH-1 viruses escaping neutralization by representative HC-84 HMAbs were similarly as described in detail elsewhere. Briefly, Huh7.5 cells (3.2×10⁴/ml) seeded 24 hours (hrs) previously in a 24-well plate were infected with 2a HCVcc (1×10⁴ FFU). Initial concentration of the neutralizing antibody employed to isolate escape HCVcc mutants was adjusted to the 50% inhibitory concentration (IC₅₀) of the antibody against the 2a HCVcc. Infectious virus was first incubated with the selection antibody for 1 hr at 37° C. prior to inoculation onto naïve Huh7.5 cells. This was followed by a second incubation for 3 hrs at 37° C. before the medium was replaced with fresh medium containing the same antibody concentration. The cultures were maintained for three days in the presence of individual HC-84 antibodies or R04 (as mock human IgG selection) and the extracellular virus was harvested for the next round of selection. The entire process constituted one passage of infectious virus under antibody selection. At each antibody concentration, the virus was repeatedly passaged until the virus titer reached 1×10⁴ FFU prior to subjecting the virus to the next higher antibody concentration. The number of rounds of amplification required for this purpose varied from antibody to antibody. Starting at IC₅₀, the antibody concentration was progressively increased (0.002, 0.001, 0.005, 0.01, 0.05, 0.1, 0.5, 1, 5, 10 and 100 μg/ml). Viral growth and emergence of escape variants were monitored weekly by two-color confocal immunofluorescence microscopy staining with the respective neutralizing antibody and an anti-NS3 antibody. Viral supernatant and cells were collected weekly, and corresponding supernatants were used for escape virus amplification followed by sequencing E2 genes to map the mutations. Viral supernatants were used for neutralization studies against escape mutants and as a source of virus stock. When virus under certain antibody selection went to undetectable levels, the selection antibodies were withdrawn from the medium, and culture were continued for an additional four passages with monitoring. Confocal immunofluorescence microscopy, focus-forming unit (FFU) assay used in virus titer determination and viral yield assay were performed as previously described.

Sequence Analysis.

Total RNA or viral RNA from virus-infected cells or virus-containing culture supernatant was extracted using commercial kits (Qiagen, Valenica, Calif.). cDNA of the E2 glycoprotein was synthesized with SuperScript III reverse transcriptase (Invitrogen, Carlsbad, Calif.) by using primer p7rev CCCGACCCCTGATGTGCCAAGC in a 20-μl reaction of the manufacturer's recommended buffer. Subsequent amplification was performed in a 50-μl reaction using the Expend High Fidelity PCR system (Roche Applied Sciences, Indianapolis, Ind.) and primers E1fwd GGTCATCATAGACATCGTTAGC & p7rev CCCGACCCCTGATGTGCCAAGC. The PCR consisted of 30 cycles at 94° C. for 60 seconds, 45° C. for 60 seconds, and 72° C. for 90 seconds. A total of 2 μl of the resulting PCR product was used as template for a nested amplification, using primer pair E2F GGCACCACCACCGTTGGAGGC & E2R TGCTTCGGCCTGGCCCAACAAGAT. This second round of PCR comprised 25 cycles at 94° C. for 60 seconds, 55° C. for 60 seconds, and 72° C. for 90 seconds. In some cases, when viral titer was low and failed to amplify E2 gene, the number of PCR cycles in the nested round was increased. The PCR products were purified with the QIAquick gel extraction kit (Qiagen, Valencia, Calif.), ligated into the TOPO cloning vector (Invitrogen, Carlsbad, Calif.), and individual clones containing an insert of the expected size were sequenced in both sense and antisense strands (Elim Biopharm, Hayward, Calif.).

### Results

The majority of neutralizing antibodies identified to date bind to conserved conformational epitopes, and inhibit E2 binding to CD81. Epitope mapping of these antibodies has revealed shared contact residues at 530 and 535 which are also contact residues of E2 binding to CD81. We have previously shown different profiles of neutralization and escape among this cluster of antibodies to overlapping epitopes. Some of these antibodies are associated with a rapid neutralization escape, made possible with a single mutation. Others are associated with a much lower virus escape, where the escape mutation has a higher cost in viral fitness. Information on immunogenic clusters mediating neutralization is useful for a rational design approach in vaccine development. Modified antigens were designed for this purpose, to identify a new cluster of neutralizing antibodies.

Generation of Neutralizing Antibodies that are Outside of CD81-535 Binding Antibodies Cluster.

Antibody yeast display libraries were constructed from peripheral blood B cells isolated from an individual with chronic HCV genotype 2b infection. Approximately 100 human sera were screened for binding to HCV E2 and neutralization activity against HCV infection. This donor serum showed high serum antibody binding titer (>1:10,000) to E2 and high neutralizing activity against genotype 2a HCVcc and 1a HCVpp. VH (immunoglobulin gamma) and VL regions from total RNA of donor B cells were amplified and cloned into the yeast vector pYD2 to generate an scFv-expressing yeast display library. The final library size was 1×10⁷ individual clones. DNA sequence analysis of 71 randomly picked scFv showed that 79% contained complete open reading frames composed of 24 different VH germ line and 30 different VL germ line genes, indicating a degree of diversity comparable to those of natural human repertoires.

Antigen from HCV E2 was designed and prepared in two different sets for the selection of specific cluster anti-HCV HMAbs from immune libraries. The logic for antigen design was followings: we previously have shown that HCV E2 contains at least three immunogenic conformational domains with distinct properties and biological functions, having neutralizing domains (as domain B & C) and non-neutralizing domain (as domain A). Further epitope studies revealed that distinct but partially overlapping sets of amino acids are critical to the binding of HMAs within each domain. This knowledge provided us with a tool to engineer HCV E2 to deplete HMAbs to domain A and B during the screening, by mutating the critical shared residues in each domain respectively. Two sets of E2 antigen for domain A and B knock out were constructed by introducing mutations at positions of Y632A and D535A respectively, designated as E2-_{Y632A} and E2-_{D535A}. For a focused HMAbs selection, E2 protein could also be captured by the immobilized domain A binding antibody to block this region and present non-domain A. An engineered E2 antigen can also provide an approach for antigen design in HCV vaccine development.

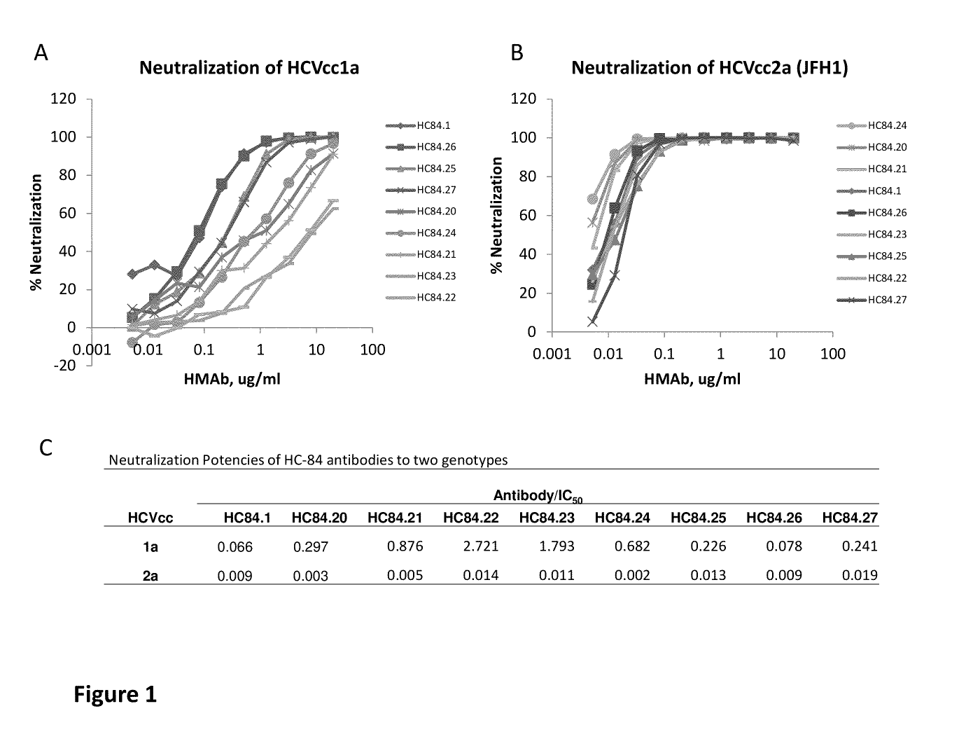

The strategy for screening unidentified HMAbs is summarized in FIG. 1. The first round of MACS selection was performed to deplete non-neutralizing anti-HCV HMAbs plus non-specific HMAbs. The immune antibody yeast display library was incubated with soluble E2-_{Y632A} protein immobilized on immunobeads. A second round of domain A depletion was carried out with soluble E2-_{Y632A} protein, and the bound HMAbs were separated by FACS. The next round of FACS selection was performed for domain B depletion by incubating the collected non-domain A fraction with soluble E2-_{D535A} protein. A total of 300 monoclonal scFv yeast were screened for binding activity to HCV E2. Figure prints and DNA sequence analysis from the binding scFv yeast identified 75 unique scFv (˜25%). All monoclonal yeast antibodies were demonstrated to specifically bind to HCV E2, but not to antigen control. We next tested the ability of these scFv to bind to soluble E2 derived from different six geno- and sub-genotypes, 1a, 1b, 2a, 2b, 3a, 4, 5 and 6. A final 9 scFv showed a broad breadth of binding, with unique sequence combinations of heavy and light chain CDR1, 2 and 3 regions. The antibodies were reformatted as IgG1 molecules and transiently expressed, designated as HC-84.1; HC-84.20; HC-84.21; HC-84.22; HC-23; HC-84.24; HC-84.25; HC-84.26 and HC-84.27. All the HMAbs were produced in serum-free medium at concentrations ranging from 30 to 120 μg/ml and further purified.

Breadth of Neutralization Against Different HCVcc Genotypes and Subtypes.

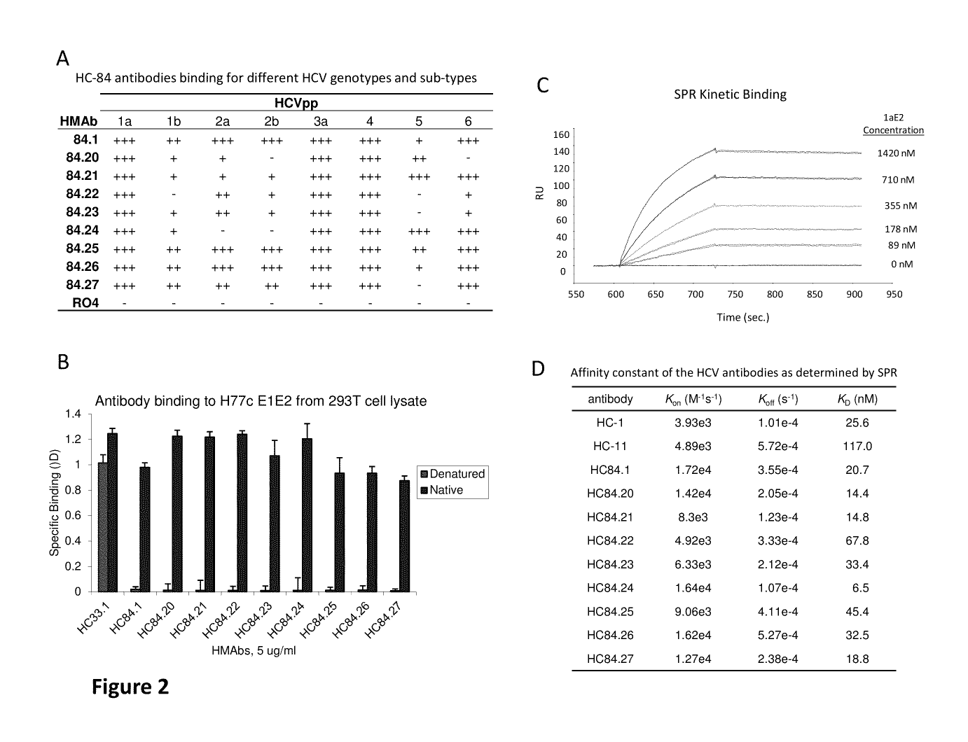

Since we implemented a bias screening strategy to focus on neutralizing HMAbs outside of domain B, we next investigated the neutralization activities of these HMAbs as one of the key functional activities (FIG. 2). The purified IgG1 HMAbs were first assayed for neutralization potency at 20 μg/ml of HMAb against 10⁵ FFU/ml of genotype 2a and 1a HCVcc, with each HMAb neutralizing both genotypes as expected. Seven of the 9 HMAbs reached the end point of 100% neutralization with two of the 9 HMAbs reached the end point of 68% neutralization at concentrations of <15 μg/ml against 1a HCVcc, whereas all 9 HMAbs reached the end point of 100% neutralization at a concentration of <20 μg/ml against 2a HCVcc.

The panel of 9 HMAbs was then tested for neutralizing activity against 2a JFH-1 HCVcc and multiple JFH-1 HCVcc bearing Core-NS2 from the genotype strains: 1a, 1b, 2a, 2b, 3a, 4, 5, 6 and 7 at three concentrations of 1, 10 and 50 ug/ml (FIG. 2B). All 9 HMAbs efficiently neutralized the genotypes 1a, 2a, 3a and 4. Of note, 1a was homologous to the strain used to screen the HMAbs. 8 of the 9 HMAbs neutralized the genotypes 1 b and 6; 7 of the 9 HMAbs neutralized the genotype 2b and 4 of the 9 HMAbs neutralized the genotype 5. Overall, a majority of the HMAb showed an a broad breadth of neutralization activity.

The 50% neutralization concentrations were further determined against two viral genotypes by FFU reduction assay after titration of purified IgG1 HC-84 HMAbs at a range from 0.001-100 μg/ml before Huh7.5 cell infection (FIG. 2A). The HMAbs have been ranked in the figures on the basis of the concentration required to reach 50% of maximal neutralization calculated by nonlinear regression. HC-84 HMAbs neutralized 1a HCVcc with IC₅₀ ranging from 0.066-2.721 μg/ml and neutralized 2a HCVcc with IC₅₀ ranging from 0.002-0.019 μg/ml. The level of neutralizing activity of HC-84 panel shows significantly greater potency than CBH-panel (as CBH-5) or HC-panel (such as HC-1 and HC-11) in neutralizing 1a and 2a HCVcc, in which case 50% inhibition was not observed at <100 μg/ml for 1a HCVcc.

Binding Activity and Affinity Determination of the Neutralizing HC-84 HMAbs.

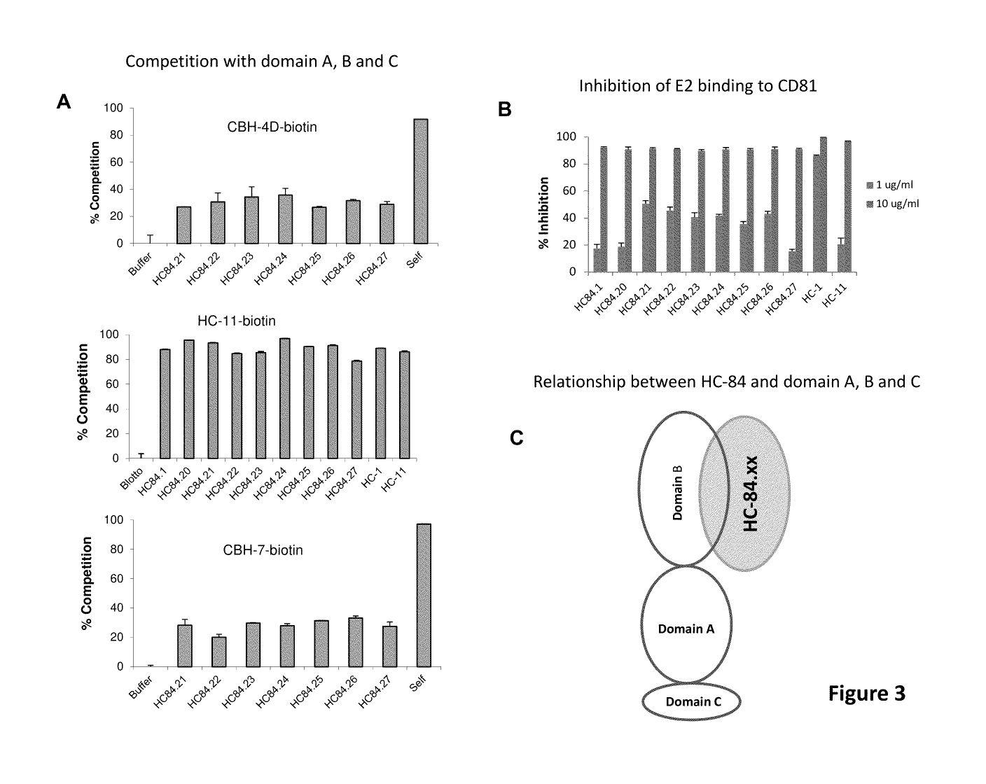

The neutralization activities demonstrated that HC-84 HMAbs are not domain A antibodies. To further evaluate the efficiency of the bias screening strategy, we tested the ability of IgG1 HMAb binding to E2 and E2_{D535A} mutant by ELISA (FIG. 3A). As expected all 9 HMAbs bind to both forms of E2, whereas the domain B antibody control HC-1 did not. This suggests that the modified antigens had selective influence on the outcome of the antibody screen.

The ability of these IgG1 HMAbs to bind to soluble E2 derived from different six geno- and sub-genotypes were examined again (FIG. 3B), the nine HC-84 HMAbs bound to E2 of all six genotypes and two subtypes, whereas the isotype-matched control RO4, a HMAb to a CMV specific protein, did not. Although scFv are five times smaller than the reformatted IgG1, no differences in the binding pattern was observed. All nine antibodies were able to immunoprecipitate genotype 1a E2 (FIG. 3C), but did not detect E2 under reducing conditions by either ELISA (FIG. 3D) or Western blot analysis, indicating that the HC-84 HMAbs are to conformational epitopes on HCV E2 glycoprotein. HC-33.1, a linear antibody against HCV E2 glycoprotein, was used as a positive control in FIG. 3C.

We further studied the binding affinity of the HC-84 HMAbs to 1a E2 protein. The binding activities were first determined by ELISA titration on soluble is E2 antigen to obtain approximate kd value range. Affinity determination was then performed by SPR, a representative tracing for antibody HC-84.1 is shown in FIG. 3E. The kₒₙ, k_{off}, and absolute K_{D} values were calculated. The antibody binding affinities of HC-84 HMAbs to 1a E2 protein varied over a 1-log value, ranging from 6.5 to 67.8 nM of K_{d} values.

HC-84 Related Antibodies Overlap but Distinct from Domain B Antibodies.

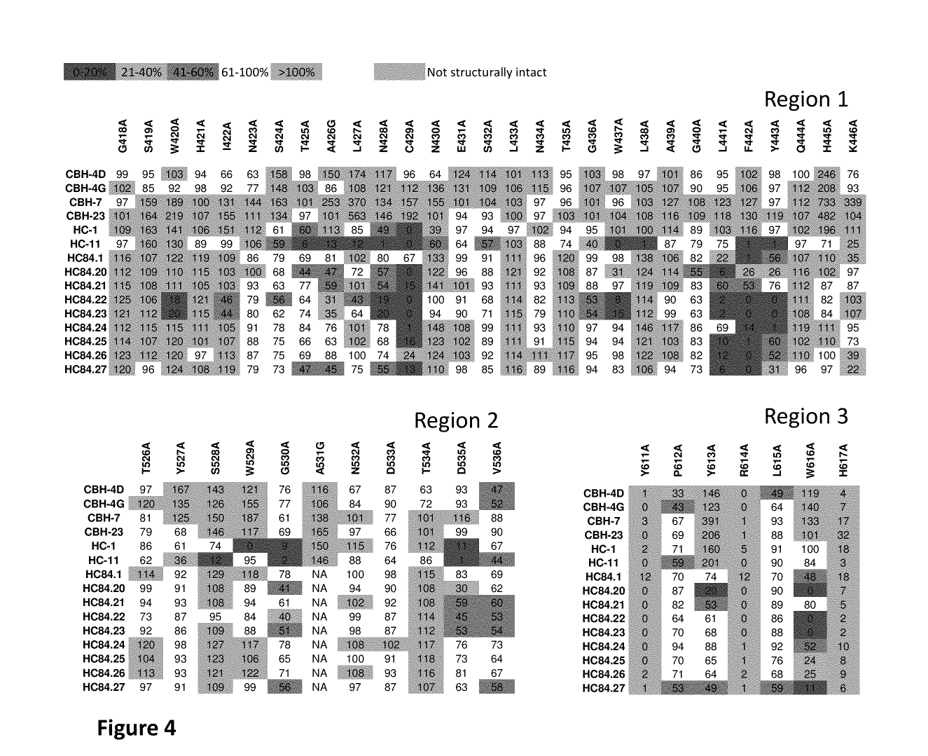

HCV E2 contains at least three immunogenic conformational domains with distinct properties and biological functions. Each domain contains multiple overlapping epitopes having similar properties and function. Since HC-84 HMAbs showed different properties in their neutralization and binding to E2 variants from existing antibodies representing each domains, we next investigated their spatial proximity of each of the conformational epitopes recognized by the nine antibodies to the other epitopes represented by the three domains, and further to themselves on E2 glycoprotein. We carried out a competition analysis using representative biotin-labeled domain A (CBH-4D), B (CBH-5) and C (CBH-7) specific HMAbs, all HMAbs blocked their own binding, as expected (FIG. 4). Each of the 9 HC-84 HMAbs showed approximate 20 to 38% competition with domain A and C specific antibodies (FIGS. 4A &C) and 60 to 70% competition with CBH-5 and HC-1 (FIG. 4B), indicating that the epitopes recognized by these new antibodies overlap but are distinct from domain B antibodies. FIG. 4D lists the percentage of residual binding of biotinylated test HMAb in the presence of the competing HMAb.

Since domain B antibodies have shown a wild range of amino acids clusters in their epitopes, to further define whether HC-84 HMAbs can be placed as a new domain or as a sub-group of domain B, all 6 known domain B-specific antibodies and 9 HC-84 antibodies were tested by cross-competition and their degree of relatedness plotted in a hierarchical fashion in a dendrogram (FIG. 4E). In this analysis, the most closely related antibodies among the 15 testing HMAbs are placed next to one another, as determined by an algorithm described previously. After the two closest related antibodies (HC-84.25 and HC-84.26) are placed, their cross-competition average is then used to identify the next closest related antibody (HC-84.27) to this pair until all antibodies are assigned (FIG. 4D). The small distance apparent between the 6 domain B HMAbs point to a series of overlapping epitopes. For HC-84 antibodies HC-84.20 is most closely related to HC84.22, and HC-84.25, 0.26 and 0.27 are more closely related to each other as a subgroup. CBH-84.21 is closer to HC-84.22 and 0.23 than to HC-84.24.

Since the HC-84 HMAbs showed binding properties that are similar to domain B antibodies, we examined their neutralization mechanism in comparison to domain B antibodies. The mechanism of neutralization with domain B HMAbs described previously is by inhibiting the binding of E2 to CD81. This was studied with HC-84 HMAbs in a CD81 capture assay. As shown in FIG. 4D, preincubation of E2 glycoproteins with 1 and 10 μg/ml of each HC-84 HMAb or CBH-5 and HC-11 reduced up to over 90% E2 binding to CD81 in a does depend manner, compared to the RO4 negative control. Similar to other domain B HMAbs described previously, the HC HMAbs appear to neutralize HCV by blocking E2 binding to CD81. Together these data suggest that HC-84 antibodies represent a new subset of domain B antibody, designated as domain B′. This subset has similar binding, neutralization properties and function to domain B, but in a residue D535 independent manner. No new domain A and C binding HMAbs were isolated in this screening. The data further suggests that residues outside of aa535 region might be utilized by this subset of domain B antibodies to engage in blocking E2-CD81 interaction in viral entry process.

Epitope Mapping HC-84 HMAbs, their Effect on Viral Infectivity and Correlation to CD81 Binding.

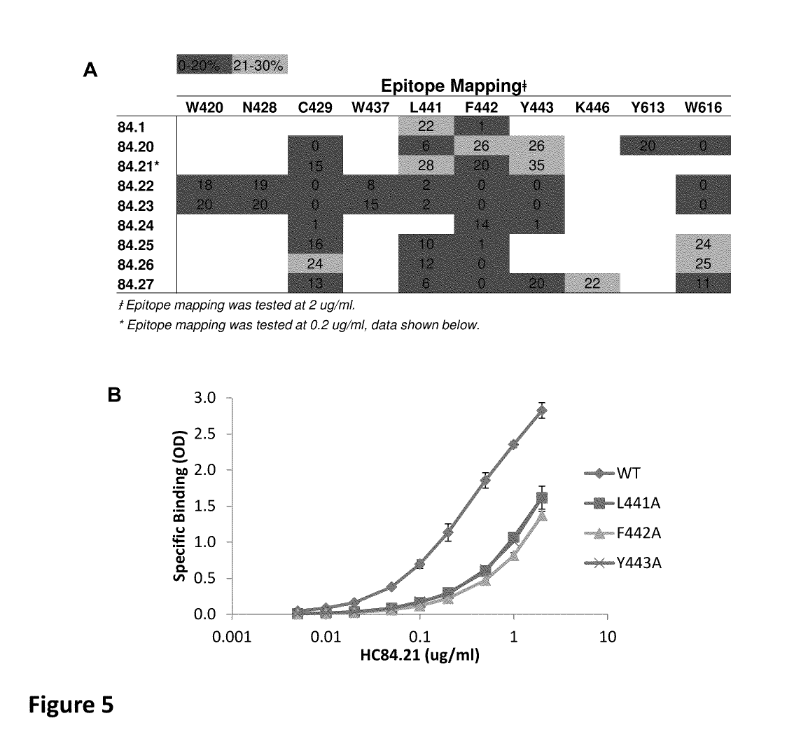

To assess which of the HC-84 HMAbs targeting region(s) of E2 involved in CD81 binding, epitope mapping was performed by conventional alanine scan binding in defined regions. Flow analysis was performed on a full length E2 mutation library (FIG. 5). Three separate segments of E2 (418-446, 526-536 and 611-616, shown as region 1, 2 and 3) were initially selected for this study based on earlier reports that some of individual residues within these regions are essential for E2 binding to CD81. Although the competition pattern with HC-11 in association with binding to E2_{D535A} mutant pointed to region 425-443 but not region 529-535, HC-84 HMAbs epitopes in other residues within region 500 could not be ruled out.

A series of alanine substitutions of residues in the H77c 1a HCVpp covering the three regions were constructed by site-directed mutagenesis. Binding by each HC-84 antibody to these proteins was examined by ELISA using lysates of transiently transfected HEK293 cells. The results were normalized according to the E2 abundance in each lysate, as determined by the binding of a non-neutralizing HMAb, CBH-17, to a linear E2 epitope. Because CBH-17 does not cross-compete with domain B antibodies, the contact residues of this antibody should not be involved in the domain B epitopes. To confirm that the E2 conformational structure was not altered with each alanine substitution, antibodies representing domain A (CBH-4G) and domain C (CBH-7) binding were also measured since they have minimal to no cross-reactivity to domain B antibodies. Thus, a substitution that results in a reduction in binding to testing antibodies as well as to either or both CBH-4G and CBH-7 was interpreted as having a global effect on E2 structure, instead of being specific for the HC-84 antibody epitope. Specific contact residues were defined as substitutions leading to 80°/o binding reduction. As shown in FIG. 5, alanine scanning of the three regions of E2 revealed that 8 contact residues 420, 428, 437, 441, 442, 443, 613 and 616 were HC-84 HMAbs-specific in regions 1 and 3, encompassing 418-446 and 611-616. HC-84.21 is an exception, although it showed similar binding and neutralization properties and close relatedness to the HC-84 antibodies by competition study, no single residue could be evidently identified as its epitope by the definition we used.

The HC-84 epitopes were further confirmed by screening the full length E2 alanine substitutions displayed on yeast surface by flow analysis. The expression of the mutant E2 protein was detected by V5-mab labeled with Alex-488 and integrity of E2 structure was assessed by the HMAbs representing domains A (CBH-4B) and C (CBH-7) labeled with Alexa-647. The expression positive yeast populations were used for selection of individual clones that bound to CBH-4B and CBH-7, but lost their ability to bind to HC-84 HMAbs. The data were broadly compatible with the HC-84 epitopes as defined above by alanine substitutions screening. No additional mutated residuals showed significant reduction (≧20% over wt binding value) for HC-84 HMAbs binding to E2. In agreement, no residues could be clearly identified as epitopes of HC-84.21. It is possible that the binding pocket of HC-84.21 is more flexible to the side-chain changes introduced by alanine than the others from the HC-84 antibodies. Further summary of contact residues for each HC-84 antibody are shown in FIG. 5.

Together, the study showed that the shared contact residues between HC-84 antibodies and domain B antibodies are 428, 437, 442 and 443 and the unique residues for HC-84 antibodies are 420, 441 and 446 in E2 region aa 420-446. Most of the epitopes of HC-84 HMAbs, HC-84.20, 84.22, 84.23, 84.25, 84.26 and 84.27, also comprise aa616 with HC-84.20 contains residual 613 in addition. Indeed, none of the epitopes contain residues 530 and 535 as in the epitopes of domain B antibodies, which are critical residues for E2 binding to CD81. Consequently, in relationship to the structural model of HCV E2 (7), the sub group of domain B, HC-84 HMAbs, spans between domain I and III whereas domain B antibodies are exclusively located within domain I. The data validated the efficiency of a screening strategy employing rational designed screening antigens.

Sequence alignment of the 10 amino acids encompassed in HC-84 antibody epitopes on E2 with other HCV sequences in HCV database revealed that seven residues (420, 428, 429, 441, 443, 613 and 616) are at 100% conservation in all the genotype and sub-genotypes. For the remaining three residues the sequence analysis showed that residue 437, specific to epitopes of HC-84.22 and 0.23, W437 is used exclusively in genotype 1, over 99% is used in genotype 3 with F437 side chain change at frequency <1% but F437 is used predominantly in genotype 2, 3, 5 and 6, indicating residue 437 may only be tolerable to the side chain change in aromatic family; residue 442, shared in all of the epitopes of HC-84 antibodies, is at 100% conservation in genotypes 1, 2, 3 and 4 but has low frequency (F to M or L) variations in genotypes 5 and 6; and residue 446, unique to the epitope of HC-84.27, is at 100% conservation in genotypes 1, 3 and 4 but no conservation in genotypes 2, 5 and 6.

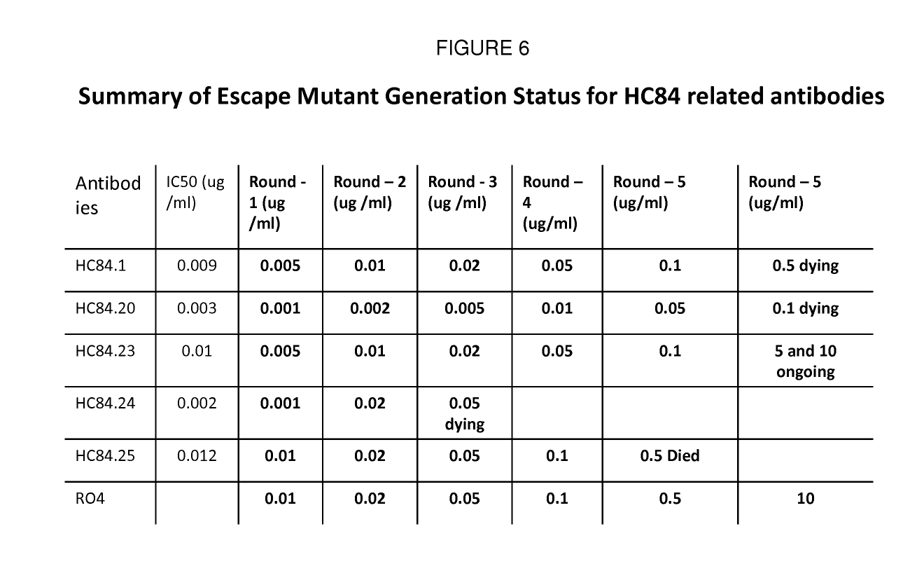

To understand viral escape from neutralizing antibodies, we investigated the correlation of the epitope conservation with their influence to virus infectivity using mutant 1a HCVpp bearing specific alanine substituted at each of these sites (FIG. 6A). Since the amino acids within HC-84 epitope are at 100% conservation in Genotype 1a, as expected, the complete or over 95% reduction in viral infectivity was observed with substitution at residues 613, 616, 420, 429, 437, 441 and 442 measured by luciferase read out, comparing with the value of wt. However, with substitution at residues 428, 443 and 446 viral infectivity was reduced by 84, 79 and 60% respectively. It is possible that alanine side chain alteration at residue 428,443 and 446 has less structural impact on the CD81 binding region.

As the majority of the amino acids within HC-84 epitopes have a significant impact on HCVpp infectivity, correlation of their role in directly binding to CD81 was assessed by CD81 pull down assay. Cell lysate of mutant 1a HCVpp bearing specific alanine substituted at each of the residue were incubated with CD81LEL-GST-glutathione-Sepharose beads and pull down was followed by Western blotting to detect E1 and E2. As shown in FIG. 6B, CD81 LEL was not able to pull down E1 and E2 for the HCVpp bearing substitution at 420, 429, 437, 441, 442, 613 and 616, indicating their involvement in E2-CD81 interaction, which is in agreement with earlier studies. E1 and E2 were precipitated with CD81 LEL for the three substitution at 428, 443 and 446 but with the reduced intensities of signals, relative to the one for wt, suggesting they may partially involve in E2-CD81 interaction and could account for the 60 to 84% reduction in viral infectivity.

The fact that all HC-84 antibodies efficiently inhibit CD81 binding to E2 implies that the involvement of critical residue(s) are likely in such order based on the degree of sharing among the 9 antibodies: F442(9/9), L441(8/9), C429(8/9), Y443(6/9), W616(6/9), W420(2/9), N428(2/9) W437(2/9), K446(1/9), Y613(1/9).

Identifying Contact Residues in HC-84 Epitopes Responsible for Virus Escape from Neutralization.

Our recent study on different neutralization escape pattern from three domain B antibodies revealed that a portion of E2 region (aa426-446) is rather flexible and prone for mutation comparing to E2 region 500, which results in neutralization escape viruses from conformational antibodies. Based on the unique composition of epitope pocket for HC-84 antibodies and in combination with their impact on HCVpp infectivity, HC-84 antibodies can be further segregated into three groups, where each group conveys a predictable outcome for neutralization escape. The first group, represented by HC-84.1, 0.25 and 0.26, may give virus no chance to escape because their epitopes encompass residues that are absolutely conserved, and mutations of these residues are lethal to viral infectivity. The second group, displayed by HC-84.20, 0.21 and 0.24, may give virus approximately 0-20% chance to escape dependent on their usage of residue Y443. The third group, represented by HC-84.22, 0.23 and 0.27, may give virus higher chance (>20%) to escape from HC-84.23 since N428 or K446 may provide additional tolerance to mutation besides Y443 within its epitope.

To assess whether the epitope analysis can be a guide in selecting the antibody that sets higher barriers for viral to escape we selected five antibodies representing three groups: two group I antibodies HC-84.1 and 0.25, 2 group II antibodies HC-84.20 and 0.24 and 1 group III antibody HC-84.23. The in vitro escape selection was designed to maximize the likelihood of escape variants by subjecting wt HCVcc to increasing concentrations of the selection antibody from IC₅₀ (μg/ml) up to 400-500 fold (μg/ml) used successfully in previously studies. At each antibody concentration, the extracellular virus was passaged repeatedly to reach a titer of 1×10⁴ FFU/ml before subjecting the virus to the next higher antibody concentration allowing minority variants to be amplified prior to the next round of selective pressure at a higher antibody concentration. As a control virus population, wt HCVcc was subjected to serial passages in increasing concentrations of R04, an isotype-matched HMAb to cytomegalovirus, to provide reference viral variants. This permitted specific discrimination between mutations introduced during long-term in vitro propagation of wt HCVcc and those mutations induced under the selective pressure of HC-84 antibodies. At the designated antibody concentrations, escape mutants were noted by a loss in specific antibody binding by indirect immunofluorescent assay, IFA (FIG. 7A). RNA from escape mutants was extracted from either cells or culture supernatants, reverse-transcribed and subcloned. Genomic residues 1491-2579 spanning the entire E2 coding region were sequenced from selected individual clones. The number of clones sequenced and analyzed per sample ranged from 20 to 60.

FIG. 6 show neutralizations escape profiles for the five tested antibodies and the control antibody, R04. The concentration of R04 was increased rapidly since this antibody has no effect on HCV and only several passages at each antibody concentration were needed to reach 10⁴ FFU/ml HCVcc. Under the selective pressure of HC-84.23 (group III in association with higher probability to have virus escape from), at least 3-4 passages at each antibody concentration from 0.1 to 1 μg/ml were required to reach 10⁴ FFU/ml. Similarly to CBH-2, this is consistent with a significant degree of HCVcc neutralization. As predicted, we observed about 10% escape viruses at 5 μg/ml.

Under the selective pressure of both group I and II selection (HC-84.1, 0.20, 0.24 and 0.25), no escape variants were isolated in two independent experiments (FIG. 7A). At low antibody concentrations of ≦5 μg/ml, virus replicated less efficiently than wt HCVcc indicating some degree of virus neutralization. When the antibody concentrations was increased to 1 μg/ml, 5 passages were required to reach 10⁴ FFU/ml. At 5 μg/ml, each round of passage led to less and less viral replication and after the fourth passage, no extracellular virus and no infected cells were observed (FIG. 7A). We attempted to rescue the virus by passaging cultured supernatant from the fourth round onto naïve Huh7.5 cells in the absence of respective antibody for four additional passages, and no detectable virus emerged from the passaged supernatants. The failure to generate HC-1-induced HCV escape mutants could be due to the viral strain employed in these studies. However, it is also possible that the group I and II epitopes are the most conserved of the antibodies tested, such that each contact residue for them is essential and the induction of escape within this epitope leads to a lethal change in virus function or structure.

1. An isolated antibody that competes for binding to HCV E2 with an antibody selected from the group comprising HC-84.1; HC-84.20; HC-84.21; HC-84.22; HC-23; HC-84.24; HC-84.25; HC-84.26 and HC-84.27.

2. The isolated antibody of claim 1, wherein the antibody is a monoclonal antibody.

3. The antibody of claim 2, wherein the antibody is a human monoclonal antibody.

4. The antibody of claim 2, wherein the antibody is a variable region fragment.

5. The antibody of claim 1, wherein the antibody comprises at least one CDR sequence as set forth in SEQ ID NO:1-54.

6. The antibody of claim 5, wherein the antibody comprises a heavy and light chain comprising, respectively, SEQ ID NO:1-3 and SEQ ID NO:28-30; the CDR sequences set forth in SEQ ID NO:4-6 and SEQ ID NO:31-33; the CDR sequences set forth in SEQ ID NO:7-9 and SEQ ID NO:34-36; the CDR sequences set forth in SEQ ID NO:10-12 and SEQ ID NO:37-39; the CDR sequences set forth in SEQ ID NO:13-15 and SEQ ID NO:40-42; the CDR sequences set forth in SEQ ID NO:16-18 and SEQ ID NO:43-45; the CRD sequences set forth in SEQ ID NO:19-21 and SEQ ID NO:46-48; the CDR sequences set forth in SEQ ID NO:22-24 and SEQ ID NO:49-52; and the CDR sequences set forth in SEQ ID NO:25-27 and SEQ ID NO:53-55.

7. The antibody of claim 6, comprising a heavy and light chain variable region sequence as set forth in SEQ ID NO:55, 64; SEQ ID NO:56, 65; SEQ ID NO:57, 66; SEQ ID NO:58, 67; SEQ ID NO:59, 68; SEQ ID NO:60, 69; SEQ ID NO:61, 70; SEQ ID NO:62, 71; and
SEQ ID NO:63, 72.

8. The antibody of claim 1, wherein the antibody neutralizes HCV in an in vitro assay.

9. The antibody of claim 1, wherein the antibody inhibits HCV infection in vivo in a subject.

10. The antibody of claim 1, wherein the antibody binds to two or more HCV genotypes of an HCV E2 protein or fragment thereof.

11. The antibody of claim 1, wherein the antibody binds to an epitope that includes one or more of HCV E2 AA420-446 or HCV E2 AA613-616, and which epitope does not include either or both of HCV E2 AA530 or HCV E2 AA535.

12. A polynucleotide encoding an antibody set forth in claim 1.

13. A cell that produces an antibody set forth in claim 1.

14. A pharmaceutical composition comprising an antibody set forth in claim 1.

15. A screening method, comprising:
mutagenizing a variable region sequence comprising one or more CDR sequences set forth in SEQ ID NO:1-54;
expressing the mutagenized sequence to provide a polypeptide product;
contacting the polypeptide with an HCV E2 antigen;
identifying those polypeptide having the desired antigen affinity or specificity.

16. A method of treating an HCV infection in a mammal comprising, administering to the mammal an antibody as set forth in claim 1, such that infectivity of cells by HCV is inhibited.

17. The method of claim 16, wherein two or more antibodies of differing specificity are administered.

18. The method of claim 16, wherein an antiviral agent is administered in combination with the antibody.

19. A method of detecting an HCV infection in a mammal comprising, contacting a body fluid of a mammal with an antibody as set forth in claim 1, and determining if binding occurs, said binding being indicative of the presence of an HCV infection.

20. An immunogenic composition comprising all or a portion of HCV E2 protein, wherein one or both of residues Y632 and D535 are masked; and
a pharmaceutically acceptable excipient.

21. The composition of claim 19, wherein the masked residues are substituted with a small uncharged amino acid.
